(19) United States
(12) Patent Application Publication
(10) Pub. No.: US 2007/0299104 A1
(43) Pub. Date: Dec. 27, 2007

(54) Tetrahydropyranyl Cyclopentyl 1-Substituted and 1,1-Disubstituted Tetrahydroisoquinoline Modulators of Chemokine Receptor Activity

(75) Inventors: Lihu Yang, Edison, NJ (US); Sander G. Mills, Scotch Plains, NJ (US); Gregori J. Moriello, Randolph, NJ (US); Deodialsingh Guiadeen, Linden, NJ (US); Kothandaraman Shankaran, Kendall Park, NJ (US)

Correspondence Address: MERCK AND CO., INC, P O BOX 2000, RAHWAY, NJ, 07065-0907, US

(73) Assignee: MERCK & CO., INC., Rahway, NJ (US)

(21) Appl. No.: 11/587,314

(22) PCT Filed: Apr. 22, 2005
(86) PCT No.: PCT/US05/13856

§ 371 Date: Oct. 23, 2006

Related U.S. Application Data
(60) Provisional application No. 60/565,382, filed on Apr. 26, 2004.

(51) Int. Cl. A61K 31/4709; A61P 19/02; C07D 217/04; C07D 217/24

(52) U.S. Cl. 514/309; 514/307; 546/141; 546/146

(57) ABSTRACT

Compounds of Formula I: I (wherein n, R₁, R₂, R₃, R₄, R₅, R₆, R₇, R₈, R₉, R₁₀, R₁₅, R₁₆, Y and Z are as defined herein) which are modulators of chemokine receptor activity and are useful in the prevention or treatment of certain inflammatory and immunoregulatory disorders and diseases, allergic diseases, atopic conditions including allergic rhinitis, dermatitis, conjunctivitis, and asthma, as well as autoimmune pathologies such as rheumatoid arthritis and atherosclerosis. The invention is also directed to pharmaceutical compositions comprising these compounds and the use of these compounds and compositions in the prevention or treatment of such diseases in which chemokine receptors are involved.

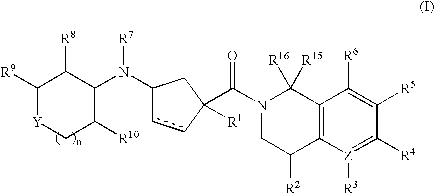

### BACKGROUND OF THE INVENTION

The chemokines are a family of small (70-120 amino acids), proinflammatory cytokines, with potent chemotactic activities. Chemokines are chemotactic cytokines that are released by a wide variety of cells to attract various cells, such as monocytes, macrophages, T cells, eosinophils, basophils and neutrophils to sites of inflammation (reviewed in Schall, Cytokine, 3, 165-183 (1991) and Murphy, Rev. Immun., 12, 593-633 (1994)). These molecules were originally defined by four conserved cysteines and divided into two subfamilies based on the arrangement of the first cysteine pair. In the CXC-chemokine family, which includes IL-8, GROα, NAP-2 and IP-10, these two cysteines are separated by a single amino acid, while in the CC-chemokine family, which includes RANTES, MCP-1, MCP-2, MCP-3, MIP-1α, MIP-1β and eotaxin, these two residues are adjacent.

The α-chemokines, such as interleukin-8 (IL-8), neutrophil-activating protein-2 (NAP-2) and melanoma growth stimulatory activity protein (MGSA) are chemotactic primarily for neutrophils, whereas β-chemokines, such as RANTES, MIP-1α, MIP-1β, monocyte chemotactic protein-1 (MCP-1), MCP-2, MCP-3 and eotaxin are chemotactic for macrophages, monocytes, T-cells, eosinophils and basophils (Deng, et al., Nature, 381, 661-666 (1996)).

The chemokines are secreted by a wide variety of cell types and bind to specific G-protein coupled receptors (GPCRs) (reviewed in Horuk, Trends Pharm. Sci., 15, 159-165 (1994)) present on leukocytes and other cells. These chemokine receptors form a sub-family of GPCRs, which, at present, consists of fifteen characterized members and a number of orphans. Unlike receptors for promiscuous chemoattractants such as C5a, fMLP, PAF, and LTB4, chemokine receptors are more selectively expressed on subsets of leukocytes. Thus, generation of specific chemokines provides a mechanism for recruitment of particular leukocyte subsets.

On binding their cognate ligands, chemokine receptors transduce an intracellular signal though the associated trimeric G protein, resulting in a rapid increase in intracellular calcium concentration. There are at least seven human chemokine receptors that bind or respond to β-chemokines with the following characteristic pattern: CCR-1 (or “CKR-1” or “CC-CKR-1”) [MIP-1αe, MIP-1β, MCP-3, RANTES] (Ben-Barruch, et al., J. Biol. Chem., 270, 22123-22128 (1995); Beote, et al, Cell, 72, 415-425 (1993)); CCR-2A and CCR-2B (or “CKR-2A”/“CKR-2A” or “CC-CKR-2A”/“CC-CKR-2A”) [MCP-1, MCP-2, MCP-3, MCP-4]; CCR-3 (or “CKR-3” or “CC-CKR-3”) [Eotaxin, Eotaxin 2, RANTES, MCP-2, MCP-3] (Rollins, et al., Blood, 90, 908-928 (1997)); CCR-4 (or “CKR-4” or “CC-CKR-4”) [MIP-1α RANTES, MCP-1] (Rollins, et al., Blood, 90, 908-928 (1997)); CCR-5 (or “CKR-5” or “CC-CKR-5”) [MIP-1α RANTES, MIP-1β] (Sanson, et al., Biochemistry, 35, 3362-3367 (1996)); and the Duffy blood-group antigen [RANTES, MCP-1] (Chaudhun, et al., J. Biol. Chem., 269, 7835-7838 (1994)). The β-chemokines include eotaxin, MIP (“macrophage inflammatory protein”), MCP (“monocyte chemoattractant protein”) and RANTES (“regulation-upon-activation, normal T expressed and secreted”) among other chemokines.

Chemokine receptors, such as CCR-1, CCR-2, CCR-2A, CCR-2B, CCR-3, CCR-4, CCR-5, CXCR-3, CXCR-4, have been implicated as being important mediators of inflammatory and immunoregulatory disorders and diseases, including asthma, rhinitis and allergic diseases, as well as autoimmune pathologies such as rheumatoid arthritis and atherosclerosis. Humans who are homozygous for the 32-basepair deletion in the CCR-5 gene appear to have less susceptibility to rheumatoid arthritis (Gomez, et al., Arthritis & Rheumatism, 42, 989-992 (1999)). A review of the role of eosinophils in allergic inflammation is provided by Kita, H., et al., J. Exp. Med. 183, 2421-2426 (1996). A general review of the role of chemokines in allergic inflammation is provided by Lustger, A. D., New England J. Med., 338(7), 426-445 (1998).

A subset of chemokines are potent chemoattractants for monocytes and macrophages. The best characterized of these is MCP-1 (monocyte chemoattractant protein-1), whose primary receptor is CCR2. MCP-1 is produced in a variety of cell types in response to inflammatory stimuli in various species, including rodents and humans, and stimulates chemotaxis in monocytes and a subset of lymphocytes. In particular, MCP-1 production correlates with monocyte and macrophage infiltration at inflammatory sites. Deletion of either MCP-1 or CCR2 by homologous recombination in mice results in marked attenuation of monocyte recruitment in response to thioglycollate injection and Listeria monocytogenes infection (Lu et al., J. Exp. Med., 187, 601-608 (1998); Kurihara et al. J. Exp. Med., 186, 1757-1762 (1997); Boring et al. J. Clin. Invest., 100, 2552-2561 (1997); Kuziel et al. Proc. Natl. Acad. Sci., 94, 12053-12058 (1997)). Furthermore, these animals show reduced monocyte infiltration into granulomatous lesions induced by the injection of schistosomal or mycobacterial antigens (Boring et al. J. Clin. Invest., 100, 2552-2561 (1997); Warmington et al. Am J. Path., 154, 1407-1416 (1999)). These data suggest that MCP-1-induced CCR2 activation plays a major role in monocyte recruitment to inflammatory sites, and that antagonism of this activity will produce a sufficient suppression of the immune response to produce therapeutic benefits in immunoinflammatory and autoimmune diseases.

Accordingly, agents which modulate chemokine receptors such as the CCR-2 receptor would be useful in such disorders and diseases.

In addition, the recruitment of monocytes to inflammatory lesions in the vascular wall is a major component of the pathogenesis of atherogenic plaque formation. MCP-1 is produced and secreted by endothelial cells and intimal smooth muscle cells after injury to the vascular wall in hypercholesterolemic conditions. Monocytes recruited to the site of injury infiltrate the vascular wall and differentiate to foam cells in response to the released MCP-1. Several groups have now demonstrated that aortic lesion size, macrophage content and necrosis are attenuated in MCP-1−/− or CCR2−/− mice backcrossed to APO-E −/−, LDL-R −/− or Apo B transgenic mice maintained on high fat diets (Boring et al. Nature, 394, 894-897 (1998); Gosling et al. J. Clin. Invest., 103, 773-778 (1999)). Thus, CCR2 antagonists may inhibit atherosclerotic lesion formation and pathological progression by impairing monocyte recruitment and differentiation in the arterial wall.

### SUMMARY OF THE INVENTION

The present invention is further directed to compounds of Formula I: 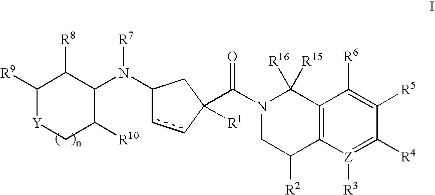
(wherein n, R¹, R², R³, R⁴, R⁵, R⁶, R⁷, R⁸, R⁹, R¹⁰, R¹⁵, R¹⁶ Y and Z are as defined herein) which are modulators of chemokine receptor activity and are useful in the prevention or treatment of certain inflammatory and immunoregulatory disorders and diseases, allergic diseases, atopic conditions including allergic rhinitis, dermatitis, conjunctivitis, and asthma, as well as autoimmune pathologies such as rheumatoid arthritis and atherosclerosis. The invention is also directed to pharmaceutical compositions comprising these compounds and the use of these compounds and compositions in the prevention or treatment of such diseases in which chemokine receptors are involved.

### DETAILED DESCRIPTION OF THE INVENTION

The present invention is directed to compounds of Formula I: 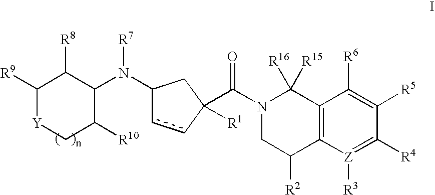
wherein:
Y is selected from —O—, —NR¹²—, —S—, —SO—, —SO₂—, and —CR¹²R¹²—, —NSO₂R¹⁴—,
—NCOR¹³—, —CR¹²COR¹¹—, —CR¹²OCOR¹³—, and —CO—;
Z is C or N;
R¹ is selected from: hydrogen, —SO₂R¹⁴, C₀₋₃alkyl-S(O)R¹⁴, —SO₂NR¹²R¹², —C₁₋₆-alkyl, —C₀₋₆alkyl-O—C₁₋₆alkyl, —C₀₋₆alkyl-S—C₁₋₆alkyl, —(C₀₋₆alkyl)-(C₃₋₇cycloalkyl)-(C₀₋₆alkyl), hydroxy, heterocycle, —CN, —
NR¹²R¹², —NR¹²COR¹³, —NR¹²SO₂R¹⁴, —COR¹¹, —CONR¹²R¹², and phenyl,
where said alkyl and cycloalkyl are unsubstituted or substituted with 1-7 substituents independently selected from: halo, hydroxy, —O—C₁₋₃alkyl, trifluoromethyl, C₁₋₃alkyl, —O—C₁₋₃alkyl, —COR¹¹, —SO₂R¹⁴, —NHCOCH₃, —NHSO₂CH₃, -heterocycle, ═O and —CN,
where said phenyl and heterocycle are unsubstituted or substituted with 1-3 substituents independently selected from: halo, hydroxy, COR¹¹, C₁₋₃ alkyl, C₁₋₃ alkoxy and trifluoromethyl;
   R² is selected from: hydrogen, hydroxy, halo, C₁₋₃alkyl unsubstituted or substituted with 1-6 substituents independently selected from fluoro and hydroxy, —NR¹²R¹², —COR¹¹, —CONR¹²R¹², —NR¹²COR¹³, —OCONR¹²R¹², —NR¹²CONR¹²R¹², -heterocycle, —CN, —NR¹²—SO₂—NR¹²R¹², —NR¹²—SO₂—R¹², —SO₂—NR¹²R¹² and ═O (oxygen connected to the ring via a double bond);
   R³ is selected from: hydrogen, C₁₋₃ alkyl unsubstituted or substituted with 1-3 fluoro, —O—C₁₋₃ alkyl, unsubstituted or substituted with 1-3 fluoro, hydroxy, chloro, fluoro, bromo, phenyl and heterocycle, when Z is C;
   R³ is O or is absent when Z is N;
   R⁴ is selected from: hydrogen, C₁₋₃alkyl unsubstituted or substituted with 1-3 fluoro, —O—C₁₋₃alkyl unsubstituted or substituted with 1-3 fluoro, hydroxy, chloro, fluoro, bromo, phenyl and heterocycle;
   R⁵ is selected from: C₁₋₆alkyl unsubstituted or substituted with 1-6 fluoro, hydroxyl or both, —O—C₁₋₆alkyl unsubstituted or substituted with 1-6 fluoro, —CO—C₁₋₆alkyl unsubstituted or substituted with 1-6 fluoro, —S—C₁₋₆alkyl unsubstituted or substituted with 1-6 fluoro, -pyridyl unsubstituted or substituted with one or more substituents selected from halo, trifluoromethyl, C₁₋₄alkyl and COR¹¹, fluoro, chloro, bromo, —C₄₋₆cycloalkyl, —O—C₄₋₆cycloalkyl, phenyl unsubstituted or substituted with one or more substituents selected from halo, trifluoromethyl, C₁₋₄alkyl, and COR¹¹, —O-phenyl unsubstituted or substituted with one or more substituents selected from halo, trifluoromethyl, C₁₋₄alkyl and COR¹¹, —C₃₋₆cycloalkyl unsubstituted or substituted with 1-6 fluoro, —O—C₃₋₆cycloalkyl unsubstituted or substituted with 1-6 fluoro, -heterocycle, —CN and —COR¹¹;
   R⁶ is selected from: hydrogen, C₁₋₃alkyl unsubstituted or substituted with 1-3 fluoro, —O—C₁₋₃alkyl unsubstituted or substituted with 1-3 fluoro, hydroxy, chloro, fluoro, bromo, phenyl and heterocycle;
   R⁷ is hydrogen or C₁₋₆alkyl unsubstituted or substituted with 1-3 substituents independently selected from: halo, hydroxy, —CO₂H, —CO₂C₁₋₆alkyl, and —O—C₁₋₃alkyl;
   R⁸ is selected from: hydrogen, C₁₋₆alkyl unsubstituted or substituted with 1-6 substituents selected from fluoro, C₁₋₃alkoxy, hydroxyl and —COR¹¹, fluoro, —O—C₁₋₃ alkyl unsubstituted or substituted with 1-3 fluoro, C₃₋₆ cycloalkyl, —O—C₃₋₆cycloalkyl, hydroxy, —COR¹¹, —OCOR¹³;
   or R⁷ and R⁸ together are C₂₋₄alkyl or C₀₋₂alkyl-O—C₁₋₃alkyl, forming a 5-7 membered ring;
   R⁹ is selected from: hydrogen, C₁₋₆alkyl unsubstituted or substituted with 1-6 substituents selected from fluoro, C₁₋₃alkoxy, hydroxyl and —COR¹¹, COR¹¹, hydroxy and —O—C₁₋₆alkyl unsubstituted or substituted with 1-6 substituents selected from fluoro, C₁₋₃alkoxy, hydroxyl and —COR¹¹;
   or R⁸ and R⁹ together are C₁₋₄alkyl or C₀₋₃alkyl-O—C₀₋₃alkyl, forming 3-6 membered ring;
   R¹⁰ is selected from: hydrogen, C₁₋₆alkyl unsubstituted or substituted with 1-6 fluoro, fluoro, —O—C₃₋₆cycloalkyl and —O—C₁₋₃alkyl unsubstituted or substituted with 1-6 fluoro;
   or R⁸ and R¹⁰ together are C₂₋₃alkyl, forming a 5-6 membered ring, where said alkyl is unsubstituted or substituted with 1-3 substituents independently selected from: halo, hydroxy, —COR¹¹, C₁₋₃alkyl and C₁₋₃alkoxy;
   or R⁸ and R¹⁰ together are C₁₋₂alkyl-O—C₁₋₂alkyl, forming a 6-8 membered ring, where said alkyl is unsubstituted or substituted with 1-3 substituents independently selected from halo, hydroxy, —COR¹¹, C₁₋₃alkyl and C₁₋₃alkoxy;
   or R⁸ and R¹⁰ together are —O—C₁₋₂alkyl-O—, forming a 6-7 membered ring, where said alkyl is unsubstituted or substituted with 1-3 substituents independently selected from halo, hydroxy, —COR¹¹, C₁₋₃alkyl and C₁₋₃alkoxy;

R¹¹ is independently selected from: hydroxy, hydrogen, C₁₋₆ alkyl, —O—C₁₋₆alkyl, benzyl, phenyl and C₃₋₆ cycloalkyl, where said alkyl, phenyl, benzyl and cycloalkyl groups are unsubstituted or substituted with 1-3 substituents independently selected from halo, hydroxy, C₁₋₃ alkyl, C₁₋₃alkoxy, —CO₂H, —CO₂—C₁₋₆ alkyl, and trifluoromethyl;

R¹² is independently selected from: hydrogen, C₁₋₆ alkyl, benzyl, phenyl and C₃₋₆cycloalkyl, where said alkyl, phenyl, benzyl and cycloalkyl groups are unsubstituted or substituted with 1-3 substituents independently selected from halo, hydroxy, C₁₋₃ alkyl, C₁₋₃ alkoxy, —CO₂H, —CO₂—C₁₋₆ alkyl and trifluoromethyl;

R¹³ is independently selected from: hydrogen, C₁₋₆ alkyl, —O—C₁₋₆alkyl, benzyl, phenyl and C₃₋₆ cycloalkyl, where said alkyl, phenyl, benzyl and cycloalkyl groups are unsubstituted or substituted with 1-3 substituents independently selected from halo, hydroxy, C₁₋₃alkyl, C₁₋₃alkoxy, —CO₂H, —CO₂—C₁₋₆ alkyl and trifluoromethyl;

R¹⁴ is independently selected from: hydroxy, C₁₋₆ alkyl, —O—C₁₋₆alkyl, benzyl, phenyl and C₃₋₆ cycloalkyl, where said alkyl, phenyl, benzyl and cycloalkyl groups are unsubstituted or substituted with 1-3 substituents independently selected from halo, hydroxy, C₁₋₃alkyl, C₁₋₃alkoxy, —CO₂H, —CO₂—C₁₋₁₆ alkyl and trifluoromethyl;

R¹⁵ is selected from: —O—C₁₋₃alkyl unsubstituted or substituted with 1-6 fluoro, hydroxy, fluoro, C₁₋₃alkyl unsubstituted or substituted with 1-6 substituents independently selected from fluoro and hydroxy, —NR¹²R¹², —COR¹¹, —CONR¹²R¹², —NR¹²COR¹³, —OCONR¹²R¹², —NR¹²CONR¹²R¹², -heterocycle, —CN, —NR¹²—SO₂—NR¹²R¹², —NR¹²—SO₂—R¹⁴, —SO₂—NR¹²R¹² and ═O where R¹⁵ is connected to the ring via a double bond;

R¹⁶ is selected from: hydrogen, fluoro, C₁₋₃alkyl unsubstituted or substituted with 1-6 substituents independently selected from fluoro and hydroxyl, or R¹⁶ is absent when R¹⁵ is connected to the ring through a double bond;

n is 0, 1 or 2;

the dashed line represents an optional single bond;

And pharmaceutically acceptable salts thereof and individual diastereomers and enantiomers thereof.

Embodiments of the present invention include compounds of formula Ia: 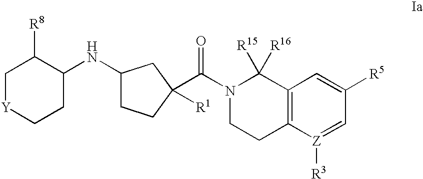
wherein R¹, R³, R⁵, R⁸, R¹⁵, R¹⁶, Z and Y are as described herein, and pharmaceutically acceptable salts and individual diastereomers and enantiomers thereof.

Embodiments of the present invention also include compounds of formula Ib: 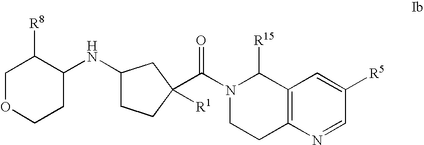
wherein R¹, R⁵, R¹⁵ and R⁸ are as described herein, and pharmaceutically acceptable salts and individual diastereomers and enantiomers thereof.

In certain embodiments of the present invention Z is N.

In certain embodiments of the present Y is —CH₂— or —O—, in particular Y is O.

In certain embodiments of the present R¹ is selected from: —C₁₋₆alkyl unsubstituted or substituted with 1-6 substituents independently selected from halo, hydroxy, —O—C₁₋₃alkyl, trifluoromethyl and —COR¹¹, —C₀₋₆alkyl-O—C₁₋₆alkyl-unsubstituted or substituted with 1-6 substituents independently selected from: halo, trifluoromethyl and —COR¹¹, and —(C₃₋₅cycloalkyl)-(C₀₋₆alkyl) unsubstituted or substituted with 1-7 substituents independently selected from halo, hydroxy, —O—C₁₋₃alkyl, trifluoromethyl and —COR¹¹. In particular, R¹ is selected from: C₁₋₆alkyl, C₁₋₆alkyl substituted with hydroxyl and C₁₋₆alkyl substituted with 1-6 fluoro.

Embodiments of the present invention also include compounds of formula I wherein one or more of R², R⁴, R⁶, R⁷, R⁹, R¹⁰ and R¹⁶ is hydrogen.

Embodiments of the present invention include compounds of formula I wherein when Z is C, R³ is hydrogen. Embodiments of the present invention also include compounds of formula I wherein when Z is N, R³ is absent.

Additional embodiments of the present invention include compounds of formula I wherein R⁵ is selected from: C₁₋₆alkyl substituted with 1-6 fluoro, —O—C₁₋₆alkyl substituted with 1-6 fluoro, chloro, bromo and phenyl. In particular, these embodiments include compounds of formula I wherein R⁵ is selected from: trifluoromethyl, trifluoromethoxy, chloro, bromo and phenyl.

Additional embodiments of the present invention include compounds of formula I wherein R⁸ is selected from: hydrogen, C₁₋₃alkyl unsubstituted or substituted with 1-6 fluoro, —O—C₁₋₃alkyl, fluoro and hydroxy. In particular, these embodiments include compounds of formula I wherein R⁸ is selected from: hydrogen, trifluoromethyl, methyl, methoxy, ethoxy, ethyl, fluoro and hydroxy.

Further embodiments of the present invention include compounds of formula I wherein R¹⁵ is selected from: fluoro, C₁₋₃ alkyl unsubstituted or substituted with 1-6 fluoro, —O—C₁₋₃ alkyl, hydroxy and ═O, where the oxygen is joined to the ring via a double bond. In particular, these embodiments include compounds of formula I wherein R¹⁵ is selected from: hydroxy, fluoro, methyl and ═O, where the oxygen is joined to the ring via a double bond;

Embodiments of the present invention include compounds of formula I wherein R¹⁶ is fluoro or hydrogen, or R¹⁶ is absent if R¹⁵ is connected to the ring via double bond.

Embodiments of the present invention include compounds of formula I wherein n is 1.

The independent syntheses of diastereomers and enantiomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

As appreciated by those of skill in the art, halo or halogen as used herein are intended to include chloro, fluoro, bromo and iodo.

As used herein, “alkyl” is intended to mean linear, branched and cyclic carbon structures having no double or triple bonds. C₁₋₈, as in C₁₋₈alkyl, is defined to identify the group as having 1, 2, 3, 4, 5, 6, 7 or 8 carbons in a linear or branched arrangement, such that C₁₋₈alkyl specifically includes methyl, ethyl, n-propyl, iso-propyl, n-butyl, iso-butyl, tert-butyl, pentyl, hexyl, heptyl and octyl. More broadly, C_{a-b}alkyl (where a and b represent whole numbers) is defined to identify the group as having a through b carbons in a linear or branched arrangement. C₀, as in C₀alkyl is defined to identify the presence of a direct covalent bond. “Cycloalkyl” is an alkyl, part or all of which which forms a ring of three or more atoms.

The term “heterocycle” as used herein is intended to include the following groups: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof.

The term “ring” is employed herein to refer to the formation or existence of a cyclic structure of any type, including free standing rings, fused rings, and bridges formed on existing rings. Rings may be non-aromatic or aromatic. Moreover, the existence or formation of a ring structure is at times herein disclosed wherein multiple substituents are defined “together”, as in “ . . . R⁸ and R⁹ together are C₁₋₄alkyl .”. In this case a ring is necessarily formed regardless of whether the term “ring” is employed.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be prepared from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media such as ether, ethyl acetate, ethanol, isopropanol, or acetonitrile are employed. Suitable salts are found, e.g. in Remington's Pharmaceutical Sciences, 17th ed., Mack Publishing Company, Easton, Pa., 1985, p. 1418.

Specific compounds within the present invention include a compound which selected from the group consisting of those compounds described in the Examples, and pharmaceutically acceptable salts thereof and individual diastereomers and enantiomers thereof.

The subject compounds are useful in a method of modulating chemokine receptor activity in a patient in need of such modulation comprising the administration of an effective amount of the compound.

The present invention is directed to the use of the foregoing compounds as modulators of chemokine receptor activity. In particular, these compounds are useful as modulators of the chemokine receptors, in particular CCR-2.

The utility of the compounds in accordance with the present invention as modulators of chemokine receptor activity may be demonstrated by methodology known in the art, such as the assay for chemokine binding as disclosed by Van Riper, et al., J. Exp. Med., 177, 851-856 (1993) which may be readily adapted for measurement of CCR-2 binding.

Receptor affinity in a CCR-2 binding assay was determined by measuring inhibition of ¹²⁵I-MCP-1 to the endogenous CCR-2 receptor on various cell types including monocytes, THP-1 cells, or after heterologous expression of the cloned receptor in eukaryotic cells. The cells were suspended in binding buffer (50 mM HEPES, pH 7.2, 5 mM MgCl₂, 1 mM CaCl₂, and 0.50% BSA or 0.5% human serum) and added to test compound or DMSO and ¹²⁵I-MCP-1 at room temperature for 1 h to allow binding. The cells were then collected on GFB filters, washed with 25 mM HEPES buffer containing 500 mM NaCl and cell bound ¹²⁵I-MCP-1 was quantified.

In a chemotaxis assay chemotaxis was performed using T cell depleted PBMC (monocytes) isolated from venous whole or leukophoresed blood and purified by Ficoll-Hypaque centrifugation followed by rosetting with neuraminidase-treated sheep erythrocytes. Once isolated, the cells were washed with HBSS containing 0.1 mg/ml BSA and suspended at 1×10⁷ cells/ml. Cells were fluorescently labeled in the dark with 2 μM Calcien-AM (Molecular Probes), for 30 min at 37° C. Labeled cells were washed twice and suspended at 5×10⁶ cells/ml in RPMI 1640 with L-glutamine (without phenol red) containing 0.1 mg/ml BSA. MCP-1 (Peprotech) at 10 ng/ml diluted in same medium or medium alone were added to the bottom wells (27 μl). Monocytes (150,000 cells) were added to the topside of the filter (30 μl) following a 15 min preincubation with DMSO or with various concentrations of test compound. An equal concentration of test compound or DMSO was added to the bottom well to prevent dilution by diffusion. Following a 60 min incubation at 37° C., 5% CO₂, the filter was removed and the topside was washed with HBSS containing 0.1 mg/ml BSA to remove cells that had not migrated into the filter. Spontaneous migration (chemokinesis) was determined in the absence of chemoattractant.

In particular, the compounds of the following examples had activity in binding to the CCR-2 receptor in the aforementioned assays, generally with an IC₅₀ of less than about 1 μM. Such a result is indicative of the intrinsic activity of the compounds in use as modulators of chemokine receptor activity.

Mammalian chemokine receptors provide a target for interfering with or promoting eosinophil and/or leukocyte function in a mammal, such as a human. Compounds which inhibit or promote chemokine receptor function, are particularly useful for modulating eosinophil and/or leukocyte function for therapeutic purposes. Accordingly, compounds which inhibit or promote chemokine receptor function would be useful in treating, preventing, ameliorating, controlling or reducing the risk of a wide variety of inflammatory and immunoregulatory disorders and diseases, allergic diseases, atopic conditions including allergic rhinitis, dermatitis, conjunctivitis, and asthma, as well as autoimmune pathologies such as rheumatoid arthritis and atherosclerosis.

For example, an instant compound which inhibits one or more functions of a mammalian chemokine receptor (e.g., a human chemokine receptor) may be administered to inhibit (i.e., reduce or prevent) inflammation. As a result, one or more inflammatory processes, such as leukocyte emigration, chemotaxis, exocytosis (e.g., of enzymes, histamine) or inflammatory mediator release, is inhibited.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

Diseases and conditions associated with inflammation and infection can be treated using the compounds of the present invention. In a certain embodiment, the disease or condition is one in which the actions of leukocytes are to be inhibited or promoted, in order to modulate the inflammatory response.

Diseases or conditions of humans or other species which can be treated with inhibitors of chemokine receptor function, include, but are not limited to: inflammatory or allergic diseases and conditions, including respiratory allergic diseases such as asthma, particularly bronchial asthma, allergic rhinitis, hypersensitivity lung diseases, hypersensitivity pneumonitis, eosinophilic pneumonias (e.g., Loeffler's syndrome, chronic eosinophilic pneumonia), delayed-type hypersensitivity, interstitial lung diseases (ILD) (e.g., idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); systemic anaphylaxis or hypersensitivity responses, drug allergies (e.g., to penicillin, cephalosporins), insect sting allergies; autoimmune diseases, such as rheumatoid arthritis, psoriatic arthritis, multiple sclerosis, systemic lupus erythematosus, myasthenia gravis, juvenile onset diabetes; glomerulonephritis, autoimmune thyroiditis, Behcet's disease; graft rejection (e.g., in transplantation), including allograft rejection or graft-versus-host disease; inflammatory bowel diseases, such as Crohn's disease and ulcerative colitis; spondyloarthropathies; scleroderma; psoriasis (including T-cell mediated psoriasis) and inflammatory dermatoses such an dermatitis, eczema, atopic dermatitis, allergic contact dermatitis, urticaria; vasculitis (e.g., necrotizing, cutaneous, and hypersensitivity vasculitis); eosinphilic myositis, eosinophilic fasciitis; cancers with leukocyte infiltration of the skin or organs. Inhibitors of chemokine receptor function may also be useful in the treatment and prevention of stroke (Hughes et al., Journal of Cerebral Blood Flow & Metabolism, 22:308-317, 2002; Takami et al., Journal of Cerebral Blood Flow & Metabolism, 22:780-784, 2002), obesity, type II diabetes, and neuropathic and inflammatory pain. Other diseases or conditions in which undesirable inflammatory responses are to be inhibited can be treated, including, but not limited to, reperfusion injury, atherosclerosis, certain hematologic malignancies, cytokine-induced toxicity (e.g., septic shock, endotoxic shock), polymyositis, dermatomyositis.

Diseases or conditions of humans or other species which can be treated with modulators of chemokine receptor function, include, but are not limited to: immunosuppression, such as that in individuals with immunodeficiency syndromes such as AIDS or other viral infections, individuals undergoing radiation therapy, chemotherapy, therapy for autoimmune disease or drug therapy (e.g., corticosteroid therapy), which causes immunosuppression; immunosuppression due to congenital deficiency in receptor function or other causes; and infections diseases, such as parasitic diseases, including, but not limited to helminth infections, such as nematodes (round worms), (Trichuriasis, Enterobiasis, Ascariasis, Hookworm, Strongyloidiasis, Trichinosis, filariasis), trematodes (flukes) (Schistosomiasis, Clonorchiasis), cestodes (tape worms) (Echinococcosis, Taeniasis saginata, Cysticercosis), visceral worms, visceral larva migraines (e.g., Toxocara), eosinophilic gastroenteritis (e.g., Anisaki sp., Phocanema sp.), and cutaneous larva migraines (Ancylostona braziliense, Ancylostoma caninum).

In addition, treatment of the aforementioned inflammatory, allergic and autoimmune diseases can also be contemplated for promoters of chemokine receptor function if one contemplates the delivery of sufficient compound to cause the loss of receptor expression on cells through the induction of chemokine receptor internalization or delivery of compound in a manner that results in the misdirection of the migration of cells.

The compounds of the present invention are accordingly useful in treating, preventing, ameliorating, controlling or reducing the risk of a wide variety of inflammatory and immunoregulatory disorders and diseases, allergic conditions, atopic conditions, as well as autoimmune pathologies. In a specific embodiment, the present invention is directed to the use of the subject compounds for treating, preventing, ameliorating, controlling or reducing the risk of autoimmune diseases, such as rheumatoid arthritis or psoriatic arthritis.

In another aspect, the instant invention may be used to evaluate putative specific agonists or antagonists of chemokine receptors, including CCR-2. Accordingly, the present invention is directed to the use of these compounds in the preparation and execution of screening assays for compounds that modulate the activity of chemokine receptors. For example, the compounds of this invention are useful for isolating receptor mutants, which are excellent screening tools for more potent compounds.

Furthermore, the compounds of this invention are useful in establishing or determining the binding site of other compounds to chemokine receptors, e.g., by competitive inhibition. The compounds of the instant invention are also useful for the evaluation of putative specific modulators of the chemokine receptors, including CCR-2. As appreciated in the art, thorough evaluation of specific agonists and antagonists of the above chemokine receptors has been hampered by the lack of availability of non-peptidyl (metabolically resistant) compounds with high binding affinity for these receptors. Thus the compounds of this invention are commercial products to be sold for these purposes.

The present invention is further directed to a method for the manufacture of a medicament for modulating chemokine receptor activity in humans and animals comprising combining a compound of the present invention with a pharmaceutical carrier or diluent.

The present invention is further directed to the use of the present compounds in treating, preventing, ameliorating, controlling or reducing the risk of infection by a retrovirus, in particular, herpes virus or the human immunodeficiency virus (HIV) and the treatment of, and delaying of the onset of consequent pathological conditions such as AIDS. Treating AIDS or preventing or treating infection by HIV is defined as including, but not limited to, treating a wide range of states of HIV infection: AIDS, ARC (AIDS related complex), both symptomatic and asymptomatic, and actual or potential exposure to HIV. For example, the compounds of this invention are useful in treating infection by HIV after suspected past exposure to HIV by, e.g., blood transfusion, organ transplant, exchange of body fluids, bites, accidental needle stick, or exposure to patient blood during surgery.

In a further aspect of the present invention, a subject compound may be used in a method of inhibiting the binding of a chemokine to a chemokine receptor, such as CCR-2, of a target cell, which comprises contacting the target cell with an amount of the compound which is effective at inhibiting the binding of the chemokine to the chemokine receptor.

The subject treated in the methods above is a mammal, for instance a human being, male or female, in whom modulation of chemokine receptor activity is desired. “Modulation” as used herein is intended to encompass antagonism, agonism, partial antagonism, inverse agonism and/or partial agonism. In an aspect of the present invention, modulation refers to antagonism of chemokine receptor activity.

The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention to the individual in need of treatment.

As used herein, the term “treatment” refers both to the treatment and to the prevention or prophylactic therapy of the aforementioned conditions.

Combined therapy to modulate chemokine receptor activity for thereby treating, preventing, ameliorating, controlling or reducing the risk of inflammatory and immunoregulatory disorders and diseases, including asthma and allergic diseases, as well as autoimmune pathologies such as rheumatoid arthritis and atherosclerosis, and those pathologies noted above is illustrated by the combination of the compounds of this invention and other compounds which are known for such utilities. For example, in treating, preventing, ameliorating, controlling or reducing the risk of inflammation, the present compounds may be used in conjunction with an antiinflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, or a cytokine-suppressing antiinflammatory agent, for example with a compound such as acetaminophen, aspirin, codeine, embrel, fentanyl, ibuprofen, indomethacin, ketorolac, morphine, naproxen, phenacetin, piroxicam, a steroidal analgesic, sufentanyl, sunlindac, tenidap, and the like. Similarly, the instant compounds may be administered with a pain reliever; a potentiator such as caffeine, an H2-antagonist, simethicone, aluminum or magnesium hydroxide; a decongestant such as phenylephrine, phenylpropanolamine, pseudophedrine, oxymetazoline, ephinephrine, naphazoline, xylometazoline, propylhexedrine, or levo-desoxy-ephedrine; an antiitussive such as codeine, hydrocodone, caramiphen, carbetapentane, or dextramethorphan; a diuretic; and a sedating or non-sedating antihistamine.

Likewise, compounds of the present invention may be used in combination with other drugs that are used in the treatment/prevention/suppression or amelioration of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention may be used. Accordingly, the pharmaceutical compositions of the present invention include those that also contain one or more other active ingredients, in addition to a compound of the present invention.

Examples of other active ingredients that may be combined with CCR2 antagonists, such as the CCR2 antagonists compounds of the present invention, either administered separately or in the same pharmaceutical compositions, include, but are not limited to: (a) VLA-4 antagonists such as those described in U.S. Pat. No. 5,510,332, WO95/15973, WO96/01644, WO96/06108, WO96/20216, WO96/22966, WO96/31206, WO96/40781, WO97/03094, WO97/02289, WO 98/42656, WO98/53814, WO98/53817, WO98/53818, WO98/54207, and WO98/58902; (b) steroids such as beclomethasone, methylprednisolone, betamethasone, prednisone, dexamethasone, and hydrocortisone; (c) immunosuppressants such as cyclosporin, tacrolimus, rapamycin and other FK-506 type immunosuppressants; (d) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, desloratadine, cetirizine, fexofenadine, descarboethoxyloratadine, and the like; (e) non-steroidal anti-asthmatics such as β2-agonists (terbutaline, metaproterenol, fenoterol, isoetharine, albuterol, bitolterol, and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); (f) non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (g) cyclooxygenase-2 (COX-2) inhibitors; (h) inhibitors of phosphodiesterase type IV (PDE-IV); (i) other antagonists of the chemokine receptors, especially CCR-1, CCR-2, CCR-3, CXCR-3 and CCR-5; 0) cholesterol lowering agents such as HMG-CoA reductase inhibitors (lovastatin, simvastatin and pravastatin, fluvastatin, atorvastatin, rosuvastatin, and other statins), sequestrants (cholestyramine and colestipol), cholesterol absorption inhibitors (ezetimibe), nicotinic acid, fenofibric acid derivatives (gemfibrozil, clofibrat, fenofibrate and benzafibrate), and probucol; (k) anti-diabetic agents such as insulin, sulfonylureas, biguanides (metformin), α-glucosidase inhibitors (acarbose) and glitazones (troglitazone and pioglitazone); (I) preparations of interferon beta (interferon beta-1a, interferon beta-10); (m) preparations of glatiramer acetate; (n) preparations of CTLA4Ig; (o) preparations of hydroxychloroquine, (p) Copaxon® (and (q) other compounds such as 5-aminosalicylic acid and prodrugs thereof, antimetabolites such as azathioprine, 6-mercaptopurine and methotrexate, and cytotoxic cancer chemotherapeutic agents.

The weight ratio of the compound of the present invention to the second active ingredient may be varied and will depend upon the effective dose of each ingredient. Generally, an effective dose of each will be used. Thus, for example, when a compound of the present invention is combined with an NSAID the weight ratio of the compound of the present invention to the NSAID will generally range from about 1000:1 to about 1:1000, or from about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin. Oily suspensions may be formulated by suspending the active ingredient in a vegetable oil, for example arachis oil, olive oil, sesame oil or coconut oil, or in a mineral oil such as liquid paraffin.

The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles.)

The pharmaceutical composition and method of the present invention may further comprise other therapeutically active compounds as noted herein which are usually applied in the treatment of the above mentioned pathological conditions.

In treating, preventing, ameliorating, controlling or reducing the risk of conditions which require chemokine receptor modulation an appropriate dosage level will generally be about 0.01 to 500 mg per kg patient body weight per day which can be administered in single or multiple doses. In certain embodiments the dosage level will be about 0.1 to about 250 mg/kg per day; or from about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions may be provided in the form of tablets containing 1.0 to 1000 milligrams of the active ingredient, or 2.0 to 500, or 3.0 to 200, particularly 1, 5, 10, 15, 20, 25, 30, 50, 75, 100, 125, 150, 175, 200, 250, 300, 400, 500, 600, 750, 800, 900, and 1000 milligrams of the active ingredient for the symptomatic adjustment of the dosage to the patient to be treated. The compounds may be administered on a regimen of 1 to 4 times per day, or once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### Schemes

Several methods for preparing the compounds of this invention are illustrated in the following Schemes and Examples. Starting materials are either commercially available or made by known procedures in the literature or as illustrated. The present invention further provides processes for the preparation of compounds of the formula I as defined above, which comprises many different sequences of assembling compounds of formula (II), formula (III) and formula (IV), or of compounds of formula (V) and formula (IV):

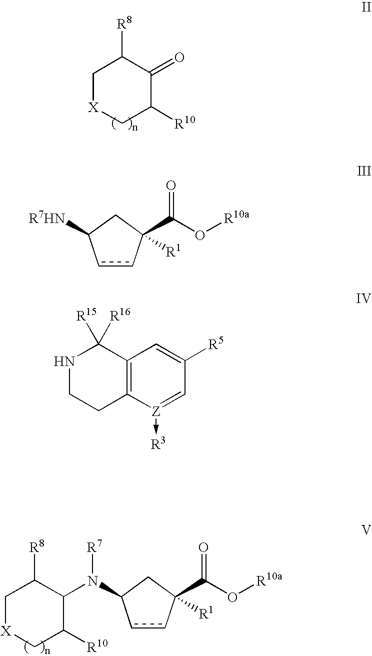

wherein R¹, R³, R⁵, R⁸, R¹⁰, and X are defined as in formula I, R^{10a} represents either a hydrogen or an alkyl group such as methyl, ethyl, t-butyl, or benzyl which serves as a protecting group, R⁷ represent either hydrogen or an amine protecting group (Greene, T; Wuts, P. G. M. Protective Groups in Organic Synthesis, John Wiley & Sons, Inc., New York, N.Y. 1991) such as Boc or trifluoroacetate. The bond between the two carbon atoms where a dashed line is shown in formula III and in formula V represent either a single or double bond as defined in formula I.

One general way of constructing target compounds of formula I utilizing Intermediates of the formulas II, III, and IV is illustrated in Scheme 1. Coupling of the acid IIIa and the amine IV under standard amide bond formation reaction conditions such as PyBrop in the presence of a base such as N,N-diisopropylethylamine and a catalyst such as DMAP gives the intermediate 1-1. Removal of the Boc protecting group yields the amine 1-2. Reductive alkylation of 1-2 with ketones II in the presence of a borohydride such as sodium triacetoxyborohydride or sodium cyanoborohydride then provides the compound of formula Ia. Note that when R⁸ or R¹⁰ are other than hydrogen, a mixture of diastereomers (Eliel, E. E., Wilen, S. H., Stereochemistry of Organic Compounds, John Wiley & Sons, Inc., New York) results from the reductive animation step. These can be separated into their components by chromatography using normal phase, reverse phase or chiral columns, depending on the nature of the separation. Compound Ia can be further elaborated to the compound of the formula I by reductive alkylation with an aldehyde or by alkylation with, for example, an alkyl halide.

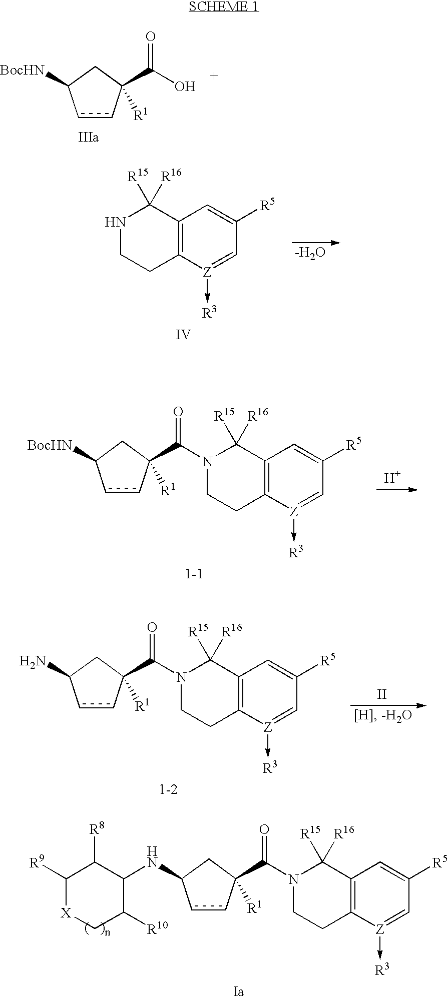

An alternative sequence of construction involving fragments of the formulas II, III, and IV is depicted in Scheme 1A. Amine IIIb is reductively alkylated with ketone II in the presence of a borohydride such as sodium triacetoxyborohydride or sodium cyanoborohydride to give secondary amine 1-3. Protection of the amine group can be accomplished using any of a number of protecting groups, including the trifluoroacetamide group (R¹²═COCF₃), which can be installed by treatment with trifluoroacetic anhydride in the presence of a base such as triethylamine. The ester functionality of the resulting compound 1-4 is then cleaved using conditions that are dependent upon the nature of R^{10a}. For example, a benzyl ester is cleaved by hydrogenolysis using a catalyst such as Pd on carbon to give the fragment of the formula V. Coupling of the acid V and the amine IV under standard amide bond formation reaction conditions such as PyBrop in the presence of a base such as N,N-diisopropylethylamine and a catalyst such as DMAP gives the intermediate 1-5. Alternatively, the acid V can be converted to its corresponding acid chloride and then treated with amine IV in the presence of a base such as triethylamine to give 1-5. Removal of the protecting group (R¹²) to give compound Ia can be achieved in various ways depending upon the nature of the protecting group. For example the trifluoroacetate group can be removed by treatment with excess sodium borohydride, or by treatment with a base such as lithium hydroxide.

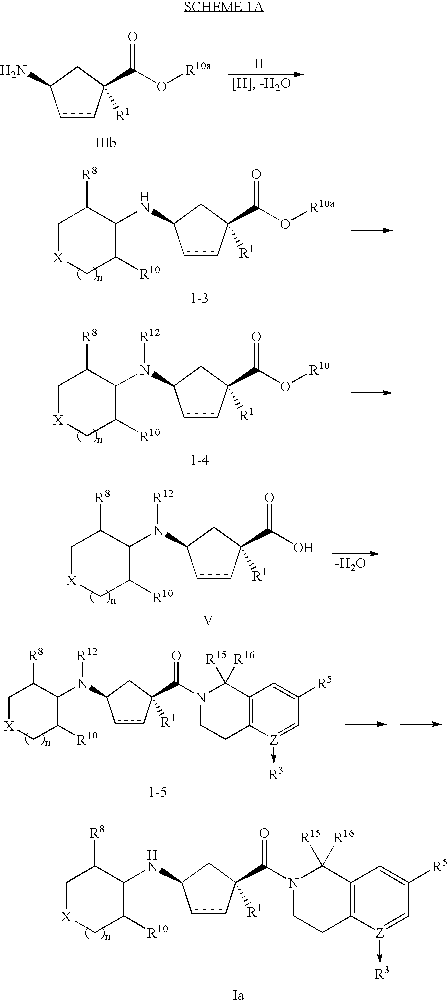

Alternatively, Intermediate 1-3 from Scheme 1A can be more directly accessed as shown in Scheme 1B. In this case amine IIIc is reductively alkylated with ketone II in the presence of a borohydride such as sodium triacetoxyborohydride or sodium cyanoborohydride to give secondary amine 1-3a. Treatment with a base such as LDA then generates the enolate of 1-3a which can be alkylated with a variety of electrophiles including but not limited to alkyl halides, aldehydes, ketones. The resulting compound 1-3 can be carried on to compounds of the formula I or Ia, using the same steps as outlined in Scheme 1A.

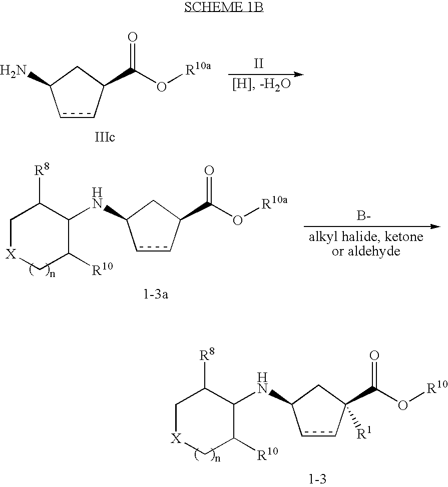

An alternate procedure for the preparation of chemokine receptor modulators of the form I is shown in Scheme 1C. According to this procedure the compounds Ib and 1c are easily prepared from intermediate 1-5 (whose preparation is described in the literature) by air oxidation.

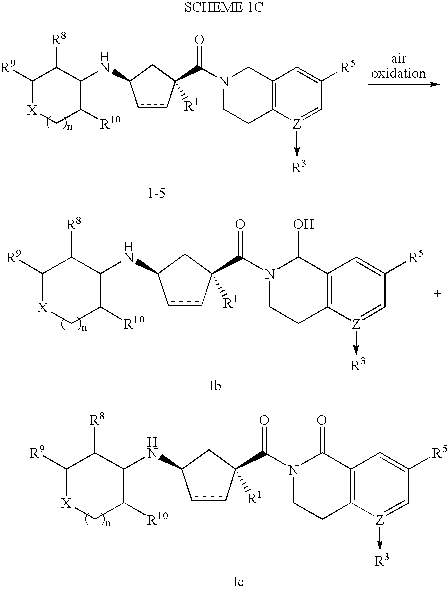

Heterocycles of the form IV can be prepared in several ways according to literature procedures or where available commercially. One such preparation is shown in Scheme 2. In this example, nitrile 2-1 is first reduced to the amine with a suitable reducing agent such as for example Raney® Ni, in methanol and ammonia. The amine (2-2) is then acylated with a suitable anhydride or acid chloride to give amide 2-3. Amide 2-3 can be cyclized to the imine 2-4 in the presence of phosphorus oxychloride and zinc chloride. The imine (2-4) can be reduced to the amine IVa using a reducing agent such as sodium triacetoxyborohydride in DCM.

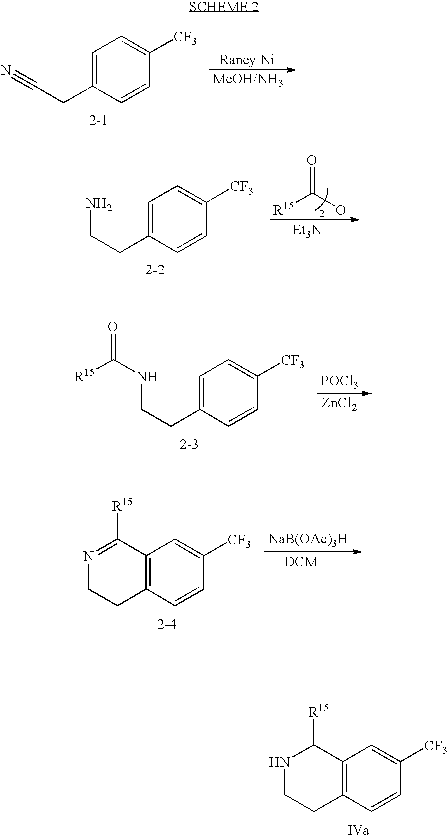

The cyclopentane core fragment III can be prepared in a number of ways. One of those is depicted in Scheme 3, 3a, and 3b. According to Scheme 3, the commercially available homochiral lactam 3-1 is hydrogenated and the saturated 3-2 is treated with di-tert-butyl dicarbonate in the presence of a suitable catalyst, e.g. N,N-dimethylamino pyridine. A base catalyzed cleavage of the amide bond in the presence of a suitable alcohol R^{10a}—OH then provides the respective ester IIIe. The BOC-protecting group is removed, preferably with an acid such as HCl in a aprotic solvent, such as dioxane, to yield the amine IIIf in the form of a salt. When this amine is mixed with benzophenone imine, the respective Schiff base IIIg is formed, which can be obtained in pure form by simple filtration to remove ammonium chloride.

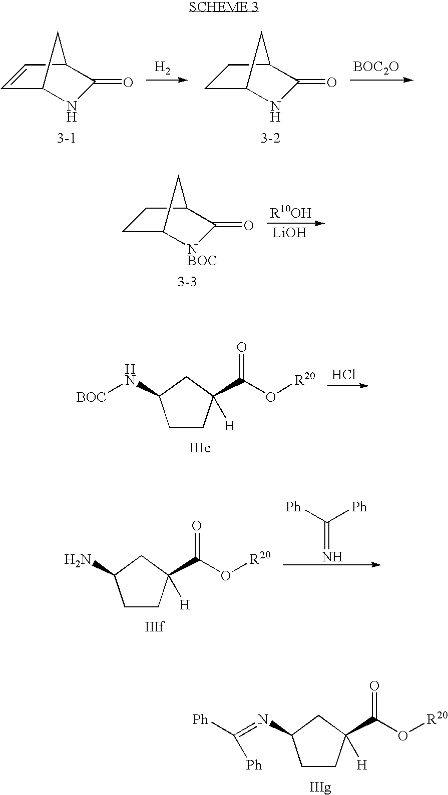

The desired cis diastereoisomers IIIh and IIIi are then treated with an acid such as HCl to aid hydrolysis of the imine group and the resulting amino group IIIj can be suitably protected e.g. in a form of a tert-butoxycarbonyl amide (Scheme 3B). The ester group present in intermediates IIIk can then be cleaved to give acid IIIl. The applied procedure depends on the nature of the ester: e.g. a benzyl ester can be cleaved by hydrogenolysis, a tert-butyl ester under acidic conditions and a alkyl ester can be hydrolyzed under either acidic or basic conditions.

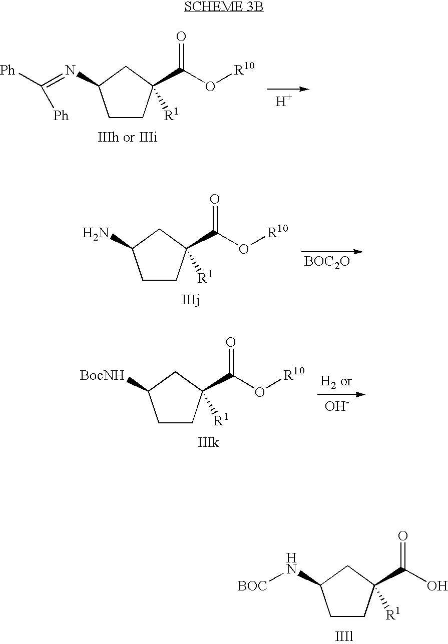

Note that Compound IIIl can be used in place of IIIa in Scheme 1, IIIj can be used in place of IIIb in Scheme 1A, and IIIf can be used in place of IIIc in Scheme 1B (the only differences being that the cyclopentane rings are defined as being fully saturated). An alternative way of preparing compounds of the type III is shown in Scheme 3C. According to this route, commercially available IIIm is converted to ester IIIn using an appropriate alcohol such as methyl or benzyl alcohol in the presence of an acid catalyst. Protection of the amine in IIIn by treatment with Boc₂O results in IIIo. Alkylation using a base such as lithium hexamethyldisilazide (LiHMDS) and an electrophile such as an alkyl halide gives IIIp, where the major diastereomer obtained is normally the cis-1,3-isomer. Separation of the cis/trans isomers can be carried out at this point or after the following step using column chromatography. If desired, hydrogenation using a catalysts such as Pd/C gives IIIq. If R¹⁰ is benzyl hydrogenation of IIIp would directly furnish IIIr. Otherwise, IIIq can be hydrolyzed using various conditions such as treatment with NaOH to give IIIr. If desired IIIr can be treated with HCl or TFA to give IIId (used in Scheme 2a).

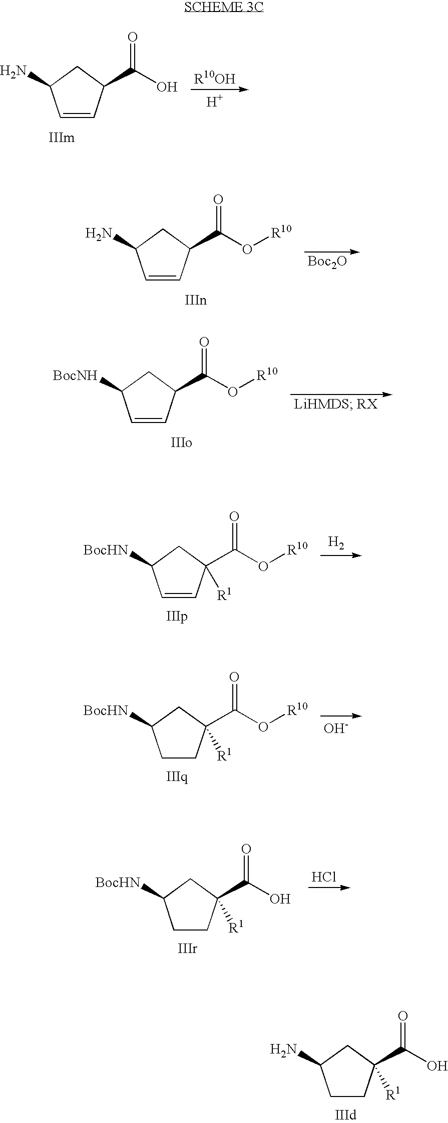

In some cases the order of carrying out the foregoing reaction schemes may be varied to facilitate the reaction or to avoid unwanted reaction products.

The following are representative procedures for the preparation of the compounds used in the following Examples or which can be substituted for the compounds used in the following Examples which may not be commercially available.

### Intermediate 1

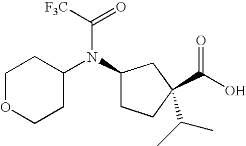 Procedure A
Step A 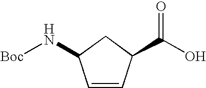
A mixture of (1R,4S)-4-amino-cyclopen-2-ene carboxylic acid (127 g, 1.0 mol), water (250 mL), sodium bicarbonate (168 g, 2.0 mol) and THF (750 mL) was stirred for 30 min, then solid Boc₂O (230 g, 1.05 mol) was added. The mixture was stirred overweekend, filtered to remove insoluble material, evaporated to remove THF, cooled at 0° C. To the residue was added 2N aq. HCl (−500 mL) until pH=3.0. The resulting precipitate was collected by filtration and washed with water, dried in vacuum overnight. The desired acid was obtained as a white solid (227g, 100%). ¹HNMR (400 MHz, CD₃OD): 5.95 (m, 1H), 5.79 (m, 1H), 4.80 (br s, 1H), 3.45 (m, 1H), 2.50 (m, 1H), 1.79 (m, 1H), 1.44 (s, 9H).
Step B 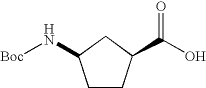
The acid (Step A, Procedure A, Intermediate 5) (227 g, 1.0 mol) and 10% Pd/C (5.0 g) in 500 mL of methanol on a Parr shaker was hydrogenated under 501b of hydrogen for one hour. The catalyst was removed by filtration and the filtrate was evaporated. The residue was dissolved in dichloromethane and dried over anhydrous sodium sulfate. After filtered, the filtrate was evaporated and dried in vacuum. The title compound was obtained as a light yellow solid (226.0 g, 99%). LC-MS for C11H19NO4 [M⁺H⁺] calculated 230, found 230.
Step C 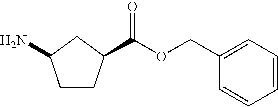
To a mechanically stirred solution of the acid (Step B, Procedure A, Intermediate 5) (226.0 g, 1.0 mol) in 500 mL of DMF was added solid potassium carbonate (210 g, 1.5 mol). The resulting mixture was stirred for 20 minutes, a neat benzyl bromide (118 mL, 1.0 mol) was added in one portion. An exothermic reaction was observed. After stirred for 3 h at RT, the entire mixture was dumped into ice-water mixture (1000 mL) was added. The crude product was extracted out with ether (2×800 mL). The combined ether layers were washed with water, dried over sodium sulfate, filtered and evaporated to offer a yellow solid. This solid was mixed with 4N HCl/dioxane (400 mL), stirred overnight and condensed. The resulting solid was collected by filtration, washed with ether and dried in vacuum. The title product was obtained as HCl salt (140g, 55%). ¹H NMR (400 MHz, CD₃OD): 5.15 (s, 2H), 3.65 (m, 1H), 3.02 (q, J=8 Hz, 1H), 2.50 (m, 1H), 2.15 (m, 1H), 2.05 (m, 2H), 1.90 (m, 1H), 1.75 (m, 1H).
Step D 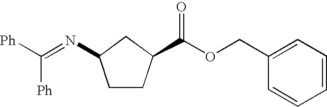
The amino benzyl ester HCl salt (Step C, Procedure A, Intermediate 5) (127 g, 0.5 mol) was suspended in 500 mL of dichloromethane. Benzophenone imine (91 g, 0.5 mol) was added. The resulting mixture was stirred overnight, filtered to remove the inorganic salt. The filtrate was washed with water and brine, dried over sodium sulfate, evaporated. The residue was dissolved in 200 mL of toluene, evaporated. This procedure was repeated once a time. The title compound (178g) was obtained as an brown oil which was used in next step without further purification. ¹H NMR (400 MHz, CDCl₃): 1.80 (m, 1H), 1.95 (m, 2H), 2.15 (m, 2H), 2.50 (m, 1H), 2.89 (m, 1H), 3.61 (m, 1H), 5.20 (s, 2H), 7.18 (d, 2H), 7.38 (m, 8H), 7.47 (m, 3H), 7.64 (d, 2H).
Step E 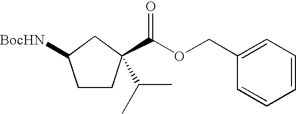
The starting Schiff base benzyl ester (Step D, Procedure A, Intermediate 5) (76.6 g, 200 mmol) in 300 mL of THF was cooled at −78° C. under nitrogen protection. While stirring, a solution of LDA (2.0 M, 110 mL, 220 mmol) in heptane was added over 20 minutes. The mixture was stirred for 30 minutes at −78° C., then a solution of 68 mL of isopropyl iodide (440 mmol) in 50 mL of THF was added, continued to stir for 30 min. The reaction temperature was raised to 0° C. by removing cooling bath. After stirred for 2 h, the entire mixture evaporated to remove THF. The residue was dissolved in ether (1000 mL), washed with water and brine, dried over sodium sulfate, evaporated. The crude product was dissolved in 500 mL of THF, mixed with 400 mL of 1N HCl, stirred for one hour, evaporated to remove THF at 50° C. The aq. solution was extracted with hexane (3×), basified with sat. aq. sodium carbonate (pH>9), mixed and stirred with a solution of Boc2O (53g) in 500 mL of dichloromethane for 30 min. The organic phase was separated and the aq. phase was extracted with dichloromethane (3×). The combined organic phases were dried over sodium sulfate and evaporated. The residue was purified on FC (10% EtOAc/hexane) to yield a mixture of the title compound as a mixture of cis and trans isomers (˜1:1, 24 g). Further purification on MPLC (5% EtOAc/Hexane) afforded single cis isomer (fast-eluted, 5.0 g) and trans isomer (slow-eluted, 4.3 g). ESI-MS calc. for C21H31NO4:361; Found: 362 (M+H).
Step F 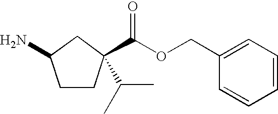
The above cis-Boc amino ester (1.25 g, 3.45 mmol) was stirred with 20 mL of 4N HCl/dioxane for one hour, evaporated and dried in high vacuum to yield the title product (1.05 g, 100%). ESI-MS calc. for C16H23NO2:261; Found: 262 (M+H).
Step G 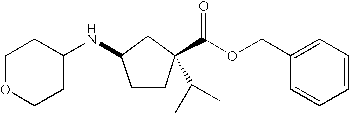
A mixture of the above amino ester (HCl salt, 1.05 g, 3.45 mmol), tetrahydro-4H-pyran-4-one (1.0 g, 10 mmol), molecular sieves (4 A, 1.0 g), DIEA (0.78 g, 6 mmol) and sodium triacetoxyborihydride (1.33 g, 6 mmol) in 30 mL of dichloromethane was stirred overnight. The reaction was quenched with sat. aq. sodium carbonate, filtered to remove insoluble material. The crude product was extracted into dichloromethane, dried over anhydrous sodium sulfate, evaporated and dried in high vacuum. The crude product was used in next step without further purification.
Step H 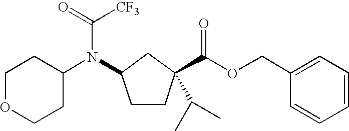
To a mixture of the crude amino ester (Step G, Procedure A, Intermediate 5) (6.85 g, 19.84 mmol), Et₃N (5.6 mL, 39.68 mmol), and DCM (50 mL), was slowly added TFAA (6.91 mL, 49.6 mmol). The reaction was stirred at room temperature for 1 hour before washed with 1N HCl and brine, dried over anhydrous MgSO₄, and concentrated in vacuo. The crude product was purified by MPLC (20/80, EtOAc/Hexanes) to yield the title compound (3.7 g, 42.2%). LC-MS for C₂₃H₃₁F₃NO₄ [M⁺H⁺] calculated 442.21, found 442.3.
Step I 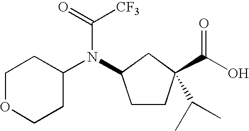
A mixture of the amide (Step H, Procedure A, Intermediate 5) (4.7 g, 10.7 mmol), 10% Pd/C (500 mg), and MeOH (50 mL) was stirred under a hydrogen balloon for 2 hours before filtered through celite and concentrated in vacuo to yield 14-C (3.92 g, 99⁺%). LC-MS for C₁₆H₂₅F₃NO₄ [M⁺H⁺] calculated 352.17, found 352.15.
Procedure B
Step A 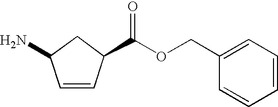
To a magnetically stirred solution of the Boc-amino acid (Step A, Procedure A, Intermediate 5) (159 g, 0.7 mol) in 500 mL of DMF was added solid potassium carbonate (138 g, 1.0 mol). The resulting mixture was stirred for 20 minutes, a neat benzyl bromide (84 mL, 0.7 mol) was added in one portion. An exothermic reaction was observed. After stirred overnight at RT, the entire mixture was dumped into ice-water mixture (1000 mL) was added. The crude product was extracted out with ethyl acetate (2×800 mL). The combined organic layers were washed with water, dried over sodium sulfate, filtered and evaporated to offer a brown oil. This material was mixed with 4N HCl/dioxane (350 mL) and stirred until no bubble evolved. 500 mL of ether was added, the precipitate was collected by filtration and washed with ether and hexane. The desired product was obtained as HCl salt (164 g, 93%). ¹H NMR (400 MHz, CD₃OD): 7.38 (m, 5H), 6.25 (m, 1H), 5.94 (m, 1H), 5.20 (s, 2H), 4.32 (br s, 1H), 3.80 (br s, 1H), 2.67 (m, 1H), 2.14 (m, 1H).
Step B 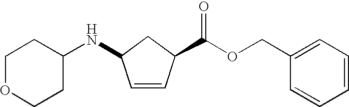
To a mixture of the amino ester HCl salt (Step A, Procedure B, Intermediate 5) (38 g, 150 mmol), tetrahydro-4-H-pyran-4-one (15 g, 150 mmol), DIEA (20.6 g, 160 mmol) and molecular sieves (4A, 20 g) in 200 mL of dichloromethane was added sodium triacetoxy borohydride (42.4 g, 200 mmol) in multiple portions. After complete addition, the mixture was stirred at RT overnight, quenched with sat. aq. sodium carbonate, filtered through celite. The crude product was extracted into dichloromethane (3×), dried over sodium sulfate and evaporated. The residue was purified on FC (10%[aq. NH4OH/MeOH 1/9]/DCM0. The desired fractions were combined and evaporated. The residue was mixed with THF and evaporated, redissolved in toluene and evaporated, dried in vacuum to yield a light brown oil (38 g, 84%). ¹H NMR (400 MHz, CDCl₃): 7.38 (m, 5H), 5.98 (m, 1H), 5.85 (m, 1H), 3.98 (m, 3H), 3.54 (m, 1H), 3.40 (m, 2H), 2.82 (m, 1H), 2.44 (m, 1H), 1.90 (m, 1H), 1.79 (m, 2H), 1.70 (m, 1H), 1.44 (m, 2H).
Step C 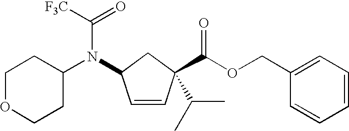
To a round flask containing solid potassium bis-(trimethylsilyl)amide (30 g, 151 mmol) under nitrogen was added 500 mL of anhydrous THF, cooled at −78° C. A solution of the amino ester (Step B, Procedure B, Intermediate 5) (38 g, 126 mmol) in 100 mL of T-BF was added in 20 minutes. The dry ice-acetone bath was changed into a dry ice-water (˜15° C.). The mixture was stirred at −15° C. for one hour, recooled at −78° C. A neat solution of isopropyl iodide (65 mL, 378 mmol) was added. The flask was placed into −15° C. bath again. After a few minutes, a large amount of white precipitate formed. The reaction mixture was stirred for additional one hour, poured into a mixture of ice and water, extracted with ether (3×). The ether layers were anti-washed with water and brine, dried over sodium sulfate and evaporated. The residue was dissolved in dichloromethane, dried over sodium sulfate again and evaporated. The residue was dried in vacuum, mixed with dichloromethane (200 mL) and cooled at 0° C. under nitrogen. To the solution was added pyridine (33 mL, 400 mmol) and trifluoroacetic anhydride (27 mL, 190 mmol) dropwise. After one hour, the reaction was quenched with water. The organic phase was separated and washed with 2N aq. HCl, water and brine. After dried over sodium sulfate and evaporated, the residue was purified on FC (20% EtOAc/hexane) to yield an light brown oil (41 g, 74%). ¹H-NMR showed a 5:1 mixture of cis/trans isomers. %). ¹H NMR (400 MHz, CDCl₃): CH═CH: C is: 6.06 (m, 1H), 5.68 (m, 1H), trans: 5.92 (m, 0.2H), 5.79 (m, 0.2H). LC-MS for C₂₃H₂₈F₃NO₄ [M+H⁺] calculated 440, found 440.
Step D 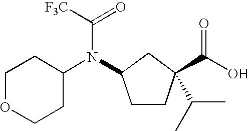
The unsaturated benzyl ester (Step C, Procedure B, Intermediates) (41 g) and 10% Pd/C (2.0 g) in ethyl acetate (100 mL) was hydrogenated on a Parr shaker under 501b of hydrogen overnight. The catalyst was removed by filtration through a pad of celite. The filtrate was evaporated and dissolved in dichloromethane, evaporated and dried in vacuum overnight. The desired acid was obtained as a gummy white solid (32.5 g, 100%). LC-MS for C16H₂₄F₃NO4 [M⁺H⁺] calculated 352, found 352.

### Intermediate 2

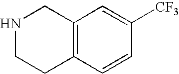 Step A

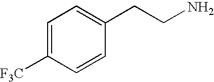

To a solution of 4-trifluoromethyl phenylacetonitrile (40 g, 215 mmol) in 2N NH3/MeOH (400 mL) was added Raney Ni (˜4.0 g). The reaction mixture was placed in a par-shaker and shook under 50 Lb pressure overnight. The solution was filtered through celite and concentrated in vacuo to yield the desired amine (38 g, 95%). ESI-MS calc. For C9H₁₀F₃N: 189; Found: 190 (M+H).
Step B 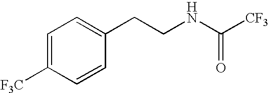
The above amine (Step A, Intermediate 2) (38 g, 200 mmol) and DIEA (52 μL, 300 mmol) were dissolved in DCM (300 mL). The solution was cooled to 0° C. before TFAA (36 mL, 250 mmol) was added slowly. The reaction mixture was stirred in the ice bath for another 10 minutes before warmed up to room temperature. The reaction was completed in 30 minutes and dumped in water and extracted with DCM (2×). The organic layer was washed with 1N HCl and saturated NaCl solution, dried over MgSO₄, and concentrated in vacuo to yield the desired amide (56 g, 98%). ESI-MS calc. For C11H9F6NO: 285; Found: 286 (M+H).
Step C 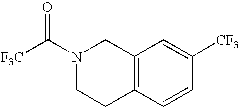
To a mixture of the amide (Step B, Intermediate 2) (73 g, 256 mmol) and paraformaldehyde (11.5 g, 385 mmol) was added 200 mL of acetic acid. The reaction mixture was stirred at room temperature for 5 min before concentrated sulfuric acid (200 mL). An exothermic reaction was observed. After 30 min, TLC showed a complete conversion. The mixture was cooled to RT before poured onto ice water (2000 mL) and extracted with EtOAc (3×500 mL). Combined organic layers were washed with water (2×), saturated NaHCO₃, and brine, dried over MgSO₄, filtered, evaporated and dried in vacuum. The desired amide (72.7 g, 96%) was obtained as a light-yellow solid. ¹H NMR (400 MHz, CDCl₃) δ 7.22 (q, J=111.67 Hz, 8.46 Hz, 1H), 7.11 (t, J=10.53 Hz, 1H), 7.03 (d, J=11.67 Hz, 1H), 4.79 (d, J=23.57 Hz, 2H), 3.91 (t, J=6.18 Hz, 1H), 3.87 (t, J=5.72 Hz, 1H), 2.97 (m, 2H). ESI-MS calc. For C12H9F6NO: 297; Found: 298 (M+H).
Step D 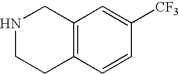
The amide (Step C, Intermediate 2) (50 g, 168 mmol) was dissolved in EtOH (200 mL) before solid K₂CO₃ (50 g, 360 mmol) and H₂O (50 mL) were added. The reaction mixture was refluxed for 15 hours before concentrated in vacuo. The concentrate was diluted with H₂O (100 mL) and extracted with DCM (5×). Combined organic layers were dried over MgSO₄, filtered, concentrated and purified on FC (10% [aq. NH4OH/MeOH 1/9]/DCM) to yield the amine (Step D, Intermediate 2)(30 g, 89%). ¹H NMR (400 MHz, CDCl₃) δ 7.11 (d, J=8.4 Hz, 1H), 7.01 (bd, J=8.4 Hz, 1H), 6.89 (s, 1H), 4.03 (s, 2H), 3.15 (t, J=6.1 Hz, 2H), 2.80 (t, J=5.6 Hz, 2H), 1.80 (s, 1H). ESI-MS calc. For C10H10F3N: 201; Found: 202 (M+H).

### Intermediate 3

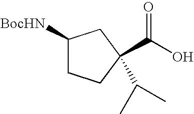 Step A 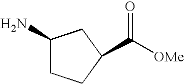
A mixture of (1S)-(+)-2-azabicyclo[2.2.1]hept-5-ene-3-one (10.3 g, 94.4 mmol) in EtOAc (200 mL) and 10% Pd/C (0.5 g) was hydrogenated at RT. After 24 h the reaction mixture was filtered and evaporated leaving behind 10.4 g (100%) of the product that was taken in 250 ml methanol and HCl (12M, 6 ml). The resultant mixture was stirred at RT, until the reaction was complete (72 h). Evaporation of methanol followed by drying under high vacuo, yielded the title compound as an off white solid (16.0 g, 96%). ¹H NMR (D₂O, 500 MHz): 3.70 (s, 3H), 3.01 (m, 1H), 2.38 (m, 1H), 2.16-1.73 (m, 6H).
Step B 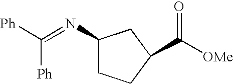
To a suspension of the Intermediate (Step A, Intermediate 4) (10.2 g, 56.8 mmol) in dry dichloromethane (200 mL) was added benzophenone imine (10.2 g, 56.8 mmol) at RT and resultant mixture was stirred for 24 h. The reaction mixture was filtered and the filtrate was evaporated, leaving behind yellow oil that was triturated with ether (100 mL), filtered and evaporated. This operation was repeated twice to ensure that the product was free of ammonium chloride impurities. The resultant oil was thoroughly dried under vacuo to yield the title compound (18.03 g, >100%) and required no further purification. ¹H NMR (CDCl₃, 500 MHz): 7.5-7.18 (m, 10H), 3.75 (m, 1H), 3.7 (s, 3H), 2.78 (m, 1H), 2.26-1.71 (m, 6H).
Step C

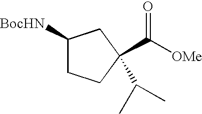

To a solution of LDA (prepared from diisopropylamine (7.7 g, 76.1 mmol) and n-BuLi (30.4 ml, 2.5 M in hexane, 76.1 mmol) in THF (120 mL) at −78° C. and the ester (Step B, Intermediate 4) was slowly added (18.0 g, 58.6 mmol). The resultant burgundy colored solution was stirred for 20 min. after which it was quenched with 2-iodopropane (14.9 g, 88 mmol). The reaction mixture was gradually warmed over 3 h to 0° C. and this temperature was maintained for additional 3 h. reaction was quenched with water and extracted with EtOAc. The organic layer was washed with water, brine, dried (anhydrous magnesium sulfate) and concentrated to yield an oil. To the solution of the crude Schiff base (20.0 g) in THF (100 mL) was added HCl (5.0 mL, 12 M) and was allowed to stir at RT for 3 h. After removal of all volatiles, the hydrochloride salt was taken up in dichloromethane (250 mL), saturated solution of sodium bicarbonate (250 mL) and Boc₂O (26.0 g, 1.4 eq.) was added. The resultant mixture was vigorously stirred overnight at RT. The organic layer was separated and washed with water, brine, dried (anhydrous magnesium sulfate) and concentrated to yield an oil. Purification by flash column chromatography (eluent: hexane: EtOAc 19:1) gave the desired product (4.91 g, 30%). ¹H NMR (CDCl₃, 500 MHz): 4.79 (br, 1H), 4.01 (m, 1H), 3.72 (s, 3H), 2.18-1.60 (m, 6H), 1.44 (s, 9H), 0.87 (d, J=6.9 Hz, 3H), 0.86 (d, J=6.9 Hz, 3H).
Step D 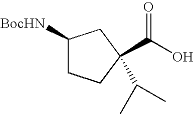
To a solution of the ester (Step C, Intermediate 4) (4.91 g, 17.2 mmol) in MeOH (100 mL) was added a solution of LiOH (3.6 g, 85 mmol) in water (20 mL) and THF (10 mL). The resultant mixture was heated at 80° C. until the reaction was complete (18 h). Methanol was removed in vacuo and the crude product was taken up with water/EtOAc (200 mL, 1:4) and cooled to 0° C. The acidity of the mixture was adjusted to pH 6. The EtOAc layer was separated, washed with water, brine, dried (anhydrous magnesium sulfate) and concentrated to yield an oil. Purification by flash column chromatography (eluent: hexane:EtOAc 1:1+2% AcOH) gave the title acid (39 g, 84%). ¹HNMR (CDCl₃, 500 MHz): 11.36 (br, 1H), 6.49 (br, 1H), 4.83 (m, 1H), 3.71 (s, 3H), 2.30-1.55 (m, 6H), 1.46 (s, 9H), 0.94 (d, J=6.9 Hz, 3H), 0.933 (d, J=6.9 Hz, 3H).

### Intermediate 4

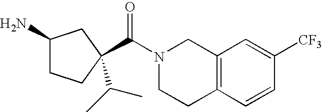 Step A 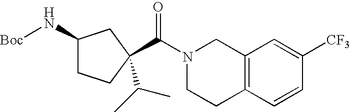
To a flask was added Boc-amino acid (Intermediate 3, 1.10 g, 4 mmol), isoquinoline hydrochloride (Intermediate 2, 0.944 g, 4 mmol), PyBrOP (1.85 g, 4 mmol), DMAP (0.29 g, 2.4 mmol), DEA (2.77 mL, 16 mmol) and DCM (20 mL). The resulting mixture was stirred for 36 h under nitrogen. The entire material was dumped onto a silica gel column and eluted with 20% EtOAc/Hexane. The desired Boc-amide was obtained as a gummy solid (1.5 g, 82%). ESI-MS calc. for C24H33F3N2O3:454; Found: 455 (M+H).
Step B 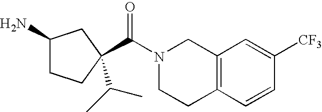
The Boc amino amide (Step A, Intermediate 6) was treated with 10 mL of 4N HCl/Dioxane for 1 h, evaporated, dried in vacuum. The intermediate 4 was obtained as a yellow solid (1.2 g). ESI-MS calc. for C19H25F3N2O:354; Found: 355 (M+H).

### Intermediate 5

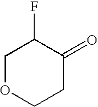 To a mixture of 5,6-dihydro-4-methoxy-2H-pyran (0.5 g, 4.4 mmol) in acetonitrile/water (15 mL, 1:1) at RT was added [1-(chloromethyl)-4-fluoro-1,4-diazoniabicyclo[2.2.2] octane bis(tetrafluoroborate)] (1.5 g, 4.4 mmol, SELECTOR™) in one lot and stirred the reaction to completion. Solid NaCl was added to the reaction mixture, then extracted with ether (4×50 mL). The ether layer dried (anhydrous magnesium sulfate) and concentrated to yield 0.34 g (65%) of the title compound that required no further purification. ¹H-NMR (500 MHz, CDCl₃): δ 4.95 (m, 1H), 4.4-4.21 (m, 2H), 3.72-3.65 (m, 2H), 2.75 (m, 2H).

### EXAMPLE 1

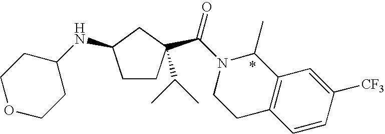 Step A 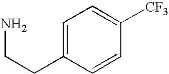
A solution of 5.0 g (27 mmol) of 4-(trifluoromethyl)phenylacetonitrile in 60 mL 2 M MeOH/NH₃ was treated with 0.7 g of Raney Nickel. The mixture was then shaken on a Parr Apparatus under 50 psi of hydrogen for 18 h. The mixture was filtered through celite and the filtrate was concentrated in vacuo to afford 4.62 g of the product amine as brown oil. ¹H NMR (CDCl₃, 400 MHz) 5:7.56-7.58, (d, 2H), 7.32-7.34 (d, 2H), 3.00 (t, 2H), 2.8 (t, 2H).
Step B 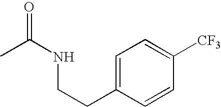
A solution of the product from Step A (2.0 g, 11 mmol) in DCM (3.0 mL) was treated with 1.10 mL (11.6 mmol) of acetic anhydride and 1.64 mL (11.6 mmol) of triethylamine at 0 C and the mixture stirred at RT for 16 h. The solvent was evaporated and the crude mixture was purified by flash chromatography, eluting with 20 to 30% EtOAc (10% MeOH)/Hexane. The title product 1.77 g (72%) was obtained as a cream solid. LC-MS for C₁₁H₁₂F₃NO [M+H]⁺ calculated 232.1, found 232.1.
Step C 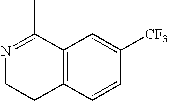
To a mixture of the product from Step B (1.50 g, 6.48 mmol) and phosphorous oxychloride (2.66 mL, 32.4 mmol) was added 1.99 g (13.0 mmol) of zinc (II) chloride and the mixture was stirred at 98° C. for 24 h and then at 120° C. for 4 days. The cool mixture was diluted with DCM and extracted with 5 N NaOH. The organic layer was washed with brine, dried (MgSO₄), and concentrated in vacuo. Flash chromatography eluting with 10 to 20% EtOAc/Hexane afforded 0.153 g of the title compound. LC-MS for C₁₁H₁₀F₃N [M+H]⁺ calculated 213.20 found 214.05.
Step D 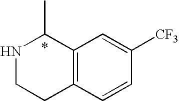
A solution of the product from Step C (0.15 g, 0.70 mmol) in DCM (5.0 mL) was treated with 0.37 g (1.75 mmol) of sodium triacetoxyborohydride and the mixture was stirred over night. The excess reducing agent was filtered off and the filtrate was concentrated. in vacuo. The crude title product (0.175 g) was obtained as an oil (racemic mixture) and was used with out further purification in the next step. LC-MS for C₁₁H₁₂F₃N [M⁺H]⁺ calculated 215.21 found 216.05.
Step E 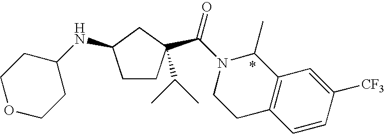
To a solution of intermediate 1 (190 mg, 0.54 mmol) in DCM under N₂ atmosphere was added 96 μL (1.1 mmol) of thionyl chloride and the mixture stirred for 2 h. The excess thionyl chloride was evaporated under N₂ atmosphere and dried under reduced pressure for 1 h. The resulting oil was re-dissolved in DCM (1 mL) and treated with the product from Step D (0.175 g, 0.81 mmol) dissolved in DCM (1 mL). Triethylamine (10 mL) was then added to the mixture and the resulting brown mixture was stirred for 24 h. The reaction was quenched with a small amount of water and the layers were separated. The organic layer was dried (MgSO₄) and concentrated in vacuo. The resulting crude mixture was dissolved in EtOH and treated with 2.0 eq of sodium borohydride and stirred for 1 h. The reaction was quenched with water, evaporated, and extracted with DCM. The organic layer was dried (MgSO₄) and concentrated in vacuo. Reverse phase IPLC purification afforded 4.2 mg of the product as a mixture of 2 stereoisomers. LC-MS for C₂₅H₃₅F₃N₂O₂ [M+H]⁺ calculated 452.55 found 453.25.

### EXAMPLE 2 and EXAMPLE 3

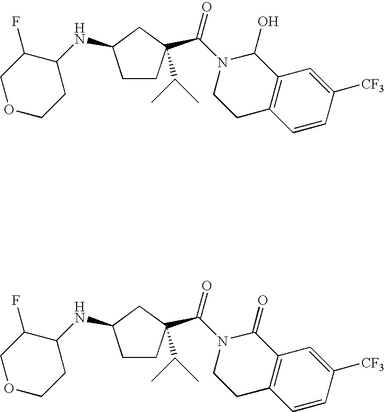 Step A 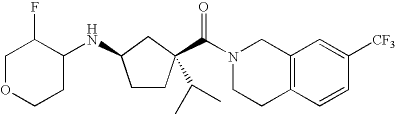
A solution of Intermediate 4 (70 mg, 0.18 mmol), Intermediate 5 (60 mg, 0.36 mmol), diisopropylethylamine (32 μL, 0.18 mmol) and crushed molecular sieves (4A, 50 mg) in dichloromethane (4 mL) was treated with sodium triacetoxyborohydride (191 mg, 0.90 mmol) and stirred at room temperature overnight. The reaction was quenched with saturated sodium bicarbonate solution (10 mL) and diluted with an additional 15 mL of DCM. The organic layer was separated and the aqueous washed with dichloromethane (2×15 mL). The organics were combined, dried over anhydrous sodium sulfate, filtered and evaporated under reduced pressure. The crude product was purified by Preparative TLC (eluant: 0.5% NH₄OH: 5% MeOH: 94.5% CH₂Cl₂) to afford the title compound as a mixture of four diastereomers. The pure single diastereoisomers were obtained by separation on chiral HPLC (ChiralPak AD, 10% ethyl alcohol in hexanes, 9.0 mL/min). ¹H NMR (500 MHz, CDCl₃) δ 7.44 (d, J=7.7 Hz, 1H), 7.38 (br s, 1H), 7.27 (dd, J=7.6 Hz, 1H), 4.80 (br d, J=17.2 Hz, 1H), 4.75 (br d, J=17.1 Hz, 1H), 3.98 (br d, J=11.5 Hz, 2H), 3.86-3.80 (m, 2H), 3.40 (app dt, J=1.4, 11.6 Hz, 2H), 3.23 (p, J=6.7 Hz, 1H), 2.93 (t, J=5.5 Hz, 2H), 2.85-2.76 (m, 1H), 2.50 (br s, 1H), 2.18-2.06 (m, 3H), 1.95 (br s, 3H), 1.84-1.78 (m, 2H), 1.66-1.57 (m, 2H), 1.51-1.33 (m, 3H), 0.92 (d, J=6.7 Hz, 3H), 0.84 (d, J=6.7 Hz, 3H). LC-MS for C₂₄H₃₂F₄N₂O₂ calculated 456.24, found [M+H]⁺ 457.3.
Step B

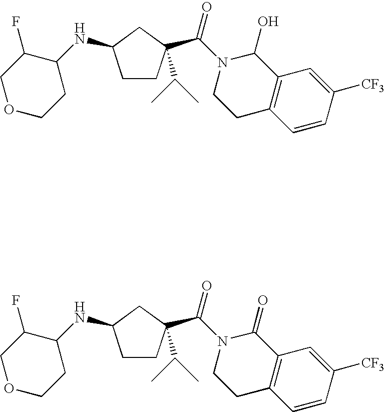

The product from Step A (221 mg) was stored at room temperature for 4 months in a scintillation vial. The oxidized products were separated by reverse phase HPLC to give 3 7.2 mg of Example 2 and 2.11 mg of Example 3 along with 13.2 of an unidentified oxidation product.

### EXAMPLE 2

¹HNMR (500 MHz, CDCl₃): δ 7.90-7.75 (m, 1H), 7.58-7.48 (m, 2H), 6.70 (br s, 1H), 6.38 (br s, 1H), 4.20-3.95 (m, 3H), 3.62-3.35 (m, 6H), 2.98-2.80 (m, 2H), 2.12 (p, J=6.6 Hz, 1H), 2.00-1.80 (m, 3H), 1.62-1.26 (m, 3H), 0.90-0.80 (m, 6H).

### EXAMPLE 3

¹H NMR (500, CDCl₃) δ 8.38 (s, 1H), 7.71 (br dd, J 2.3, 7.6 Hz, 1H), 7.38 (d, J=7.8 Hz, 1H), 6.24 (br s, 1H), 4.13-4.06 (m, 1H), 4.05-3.97 (m, 2H), 3.90 (s, 1H), 3.62 (app dt, J=2.8, 6.6 Hz, 2H), 3.51-3.40 (m, 2H), 3.10 (t, J=6.6 Hz, 2H), 2.17 (p, J=6.8 Hz, 1H), 1.91-1.84 (m, 1H), 1.82-1.56 (m, 5H), 1.42-1.36 (m, 1H), 1.32 (d, J=8.9 Hz, 1H), 1.27 (s, 1H), 1.05 (d, J=6.5 Hz, 3H), 1.03 (d, J=6.3 Hz, 3H). LC-MS for C₂₄H₃₀F₄N₂O₃ calculated 470.24, found [M+H]⁺ 471.2.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A compound of Formula I: 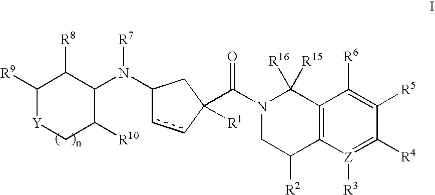
wherein:
Y is selected from —O—, NR¹²—, —S—, —SO—, —SO₂—, and —CR¹²R¹²—, —NSO₂R¹⁴—,
—NCOR¹³—, —CR¹²COR¹¹—, —CR¹²OCOR¹³—, and —CO—;
Z is C or N;
R¹ is selected from: hydrogen, —SO₂R¹⁴, C₀₋₃alkyl-S(O)R¹⁴, —SO₂NR¹²R¹², —C₁₋₆alkyl, —C₀₋₆alkyl, —O—C₁₋₆alkyl, —C₀₋₆alkyl-S—C₁₋₆alkyl, —(C₀₋₆alkyl)-(C₃₋₇cycloalkyl)-(C₀₋₆alkyl), hydroxy, heterocycle, —CN, —NR¹²R¹², —NR¹²COR¹³, —NR¹²SO₂R¹⁴, —COR¹¹, —CONR¹²R¹², and phenyl,
where said alkyl and cycloalkyl are unsubstituted or substituted with 1-7 substituents independently selected from: halo, hydroxy, —O—C₁₋₃alkyl, trifluoromethyl, C₁₋₃alkyl, —O—C₁₋₃alkyl, —COR¹¹, —SO₂R¹⁴, —NHCOCH₃, —NHSO₂CH₃, -heterocycle, ═O and —CN,
where said phenyl and heterocycle are unsubstituted or substituted with 1-3 substituents independently selected from: halo, hydroxy, COR¹¹, C₁₋₃alkyl, C₁₋₃alkoxy and trifluoromethyl;
R² is selected from: hydrogen, hydroxy, halo, C₁₋₃alkyl unsubstituted or substituted with 1-6 substituents independently selected from fluoro and hydroxy, —NR¹²R¹², —COR¹¹, —CONR¹²R¹², —NR¹²COR¹³, —OCONR¹²R¹², —NR¹²CONR¹²R¹², -heterocycle, —CN, —NR¹²—SO₂—NR¹²R¹², —NR¹²—SO₂—R¹², —SO₂—NR¹²R¹² and ═O (oxygen connected to the ring via a double bond);
R³ is selected from: hydrogen, C₁₋₃alkyl unsubstituted or substituted with 1-3 fluoro, —O—C₁₋₃alkyl, unsubstituted or substituted with 1-3 fluoro, hydroxy, chloro, fluoro, bromo, phenyl and heterocycle, when Z is C;
R³ is O or is absent when Z is N;
R⁴ is selected from: hydrogen, C₁₋₃ alkyl unsubstituted or substituted with 1-3 fluoro, —O—C₁₋₃ alkyl unsubstituted or substituted with 1-3 fluoro, hydroxy, chloro, fluoro, bromo, phenyl and heterocycle;
R⁵ is selected from: C₁₋₆alkyl unsubstituted or substituted with 1-6 fluoro, hydroxyl or both, —O—C₁₋₆alkyl unsubstituted or substituted with 1-6 fluoro, —CO—C₁₋₆alkyl unsubstituted or substituted with 1-6 fluoro, —S—C₁₋₆alkyl unsubstituted or substituted with 1-6 fluoro, -pyridyl unsubstituted or substituted with one or more substituents selected from halo, trifluoromethyl, C₁₋₄alkyl and COR¹¹, fluoro, chloro, bromo, —C₄₋₆cycloalkyl, —O—C₄₋₆cycloalkyl, phenyl unsubstituted or substituted with one or more substituents selected from halo, trifluoromethyl, C₁₋₄alkyl, and COR¹¹, —O-phenyl unsubstituted or substituted with one or more substituents selected from halo, trifluoromethyl, C₁₋₄alkyl and COR¹¹, —C₃₋₆cycloalkyl unsubstituted or substituted with 1-6 fluoro, —O—C₃₋₆cycloalkyl unsubstituted or substituted with 1-6 fluoro, -heterocycle, —CN and —COR¹¹;
R⁶ is selected from: hydrogen, C₁₋₃ alkyl unsubstituted or substituted with 1-3 fluoro, —O—C₁₋₃ alkyl unsubstituted or substituted with 1-3 fluoro, hydroxy, chloro, fluoro, bromo, phenyl and heterocycle;
R⁷ is hydrogen or C₁₋₆alkyl unsubstituted or substituted with 1-3 substituents independently selected from: halo, hydroxy, —CO₂H, —CO₂C₁₋₆alkyl, and —O—C₁₋₃alkyl;
R⁸ is selected from: hydrogen, C₁₋₆alkyl unsubstituted or substituted with 1-6 substituents selected from fluoro, C₁₋₃alkoxy, hydroxyl and —COR¹¹, fluoro, —O—C₁₋₃alkyl unsubstituted or substituted with 1-3 fluoro, C₃₋₆ cycloalkyl, —O—C₃₋₆cycloalkyl, hydroxy, —COR¹¹, —OCOR¹³;
or R⁷ and R⁸ together are C₂₋₄alkyl or C₀₋₂alkyl-O—C₁₋₃alkyl, forming a 5-7 membered ring;
R⁹ is selected from: hydrogen, C₁₋₆alkyl unsubstituted or substituted with 1-6 substituents selected from fluoro, C₁₋₃alkoxy, hydroxyl and —COR¹¹, COR¹¹, hydroxy and —O—C₁₋₆alkyl unsubstituted or substituted with 1-6 substituents selected from fluoro, C₁₋₃alkoxy, hydroxyl and —COR¹¹;
or R⁸ and R⁹ together are C₁₋₄alkyl or C₀₋₃alkyl-O—C₀₋₃alkyl, forming 3-6 membered ring;
R¹⁰ is selected from: hydrogen, C₁₋₆alkyl unsubstituted or substituted with 1-6 fluoro, fluoro, —O—C₃₋₆cycloalkyl and —O—C₁₋₃alkyl unsubstituted or substituted with 1-6 fluoro;
or R⁸ and R¹⁰ together are C₂₋₃alkyl, forming a 5-6 membered ring, where said alkyl is unsubstituted or substituted with 1-3 substituents independently selected from: halo, hydroxy, —COR¹¹, C₁₋₃alkyl and C₁₋₃alkoxy;
or R⁸ and R¹⁰ together are C₁₋₂alkyl-O—C₁₋₂alkyl, forming a 6-8 membered ring, where said alkyl is unsubstituted or substituted with 1-3 substituents independently selected from halo, hydroxy, —COR¹¹, C₁₋₃alkyl and C₁₋₃alkoxy;
or R⁸ and R¹⁰ together are —O—C₁₋₂alkyl-O—, forming a 6-7 membered ring, where said alkyl is unsubstituted or substituted with 1-3 substituents independently selected from halo, hydroxy, —COR¹¹, C₁₋₃alkyl and C₁₋₃alkoxy;
R¹¹ is independently selected from: hydroxy, hydrogen, C₁₋₆ alkyl, —O—C₁₋₆alkyl, benzyl, phenyl and C₃₋₆ cycloalkyl, where said alkyl, phenyl, benzyl and cycloalkyl groups are unsubstituted or substituted with 1-3 substituents independently selected from halo, hydroxy, C₁₋₃alkyl, C₁₋₃alkoxy, —CO₂H, —CO₂—C₁₋₆ alkyl, and trifluoromethyl;
R¹² is independently selected from: hydrogen, C₁₋₆ alkyl, benzyl, phenyl and C₃₋₆cycloalkyl, where said alkyl, phenyl, benzyl and cycloalkyl groups are unsubstituted or substituted with 1-3 substituents independently selected from halo, hydroxy, C₁₋₃ alkyl, C₁₋₃alkoxy, —CO₂H, —CO₂—C₁₋₆ alkyl and trifluoromethyl;
R¹³ is independently selected from: hydrogen, C₁₋₆ alkyl, —O—C₁₋₆alkyl, benzyl, phenyl and C₃₋₆ cycloalkyl, where said alkyl, phenyl, benzyl and cycloalkyl groups are unsubstituted or substituted with 1-3 substituents independently selected from halo, hydroxy, C₁₋₃alkyl, C₁₋₃alkoxy, —CO₂H, —CO₂—C₁₋₆ alkyl and trifluoromethyl;
R¹⁴ is independently selected from: hydroxy, C₁₋₆ alkyl, —O—C₁₋₆alkyl, benzyl, phenyl and C₃₋₆ cycloalkyl, where said alkyl, phenyl, benzyl and cycloalkyl groups are unsubstituted or substituted with 1-3 substituents independently selected from halo, hydroxy, C₁₋₃ alkyl, C₁₋₃ alkoxy, —CO₂H, —CO₂—C₁₋₆ alkyl and trifluoromethyl;
R¹⁵ is selected from: —O—C₁₋₃alkyl unsubstituted or substituted with 1-6 fluoro, hydroxy, fluoro, C₁₋₃alkyl unsubstituted or substituted with 1-6 substituents independently selected from fluoro and hydroxy, —NR¹²R¹², —COR¹¹, —CONR¹²R¹², —NR¹²COR¹³, —OCONR¹²R¹², —NR¹²CONR¹²R¹², -heterocycle, —CN, —NR¹²—SO₂—NR¹²R¹², —NR¹²—SO₂—R¹⁴, —SO₂—NR¹²R¹² and ═O where R¹⁵ is connected to the ring via a double bond;
R¹⁶ is selected from: hydrogen, fluoro, C₁₋₃alkyl unsubstituted or substituted with 1-6 substituents independently selected from fluoro and hydroxyl, or R¹⁶ is absent when R¹⁵ is connected to the ring through a double bond;
n is 0, 1 or 2;
the dashed line represents an optional single bond;
and pharmaceutically acceptable salts thereof and individual diastereomers and enantiomers thereof.

2. The compound of claim 1 of the Formula Ia: 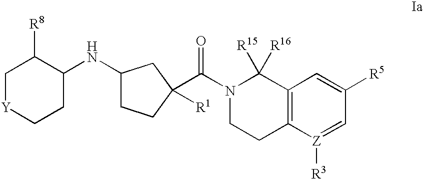
and pharmaceutically acceptable salts thereof and individual diastereomers and enantiomers thereof.

3. The compound of claim 1 of the Formula Ib: 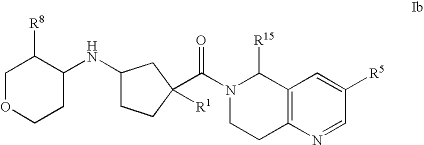
and pharmaceutically acceptable salts thereof and individual diastereomers and enantiomers thereof.

4. The compound of claim 1 wherein Z is N.

5. The compound of claim 1 wherein Y is O.

6. The compound of claim 1 wherein R¹ is selected from: —C₁₋₆alkyl unsubstituted or substituted with 1-6 substituents independently selected from halo, hydroxy, —O—C₁₋₃alkyl, trifluoromethyl and —COR¹¹, —Co-6alkyl-O—C₁₋₆alkyl-unsubstituted or substituted with 1-6 substituents independently selected from: halo, trifluoromethyl and —COR¹¹, and —(C₃₋₅cycloalkyl)-(C₀₋₆alkyl) unsubstituted or substituted with 1-7 substituents independently selected from halo, hydroxy, —O—C₁₋₃alkyl, trifluoromethyl and —COR¹¹.

7. The compound of claim 1 wherein when Z is C, R³ is hydrogen and wherein when Z is N, R³ is absent.

8. The compound of claim 1 wherein R⁵ is selected from: C₁₋₆alkyl substituted with 1-6 fluoro, —O—C₁₋₆alkyl substituted with 1-6 fluoro, chloro, bromo and phenyl.

9. The compound of claim 8 wherein R⁵ is selected from: trifluoromethyl, trifluoromethoxy, chloro, bromo and phenyl.

10. The compound of claim 1 wherein R⁸ is selected from: hydrogen, C₁₋₃alkyl unsubstituted or substituted with 1-6 fluoro, —O—C₁₋₃alkyl, fluoro and hydroxy.

11. The compound of claim 1 wherein R¹⁵ is selected from: fluoro, C₁₋₃alkyl unsubstituted or substituted with 1-6 fluoro, —O—C₁₋₃alkyl, hydroxy and ═O.

12. A compound selected from: 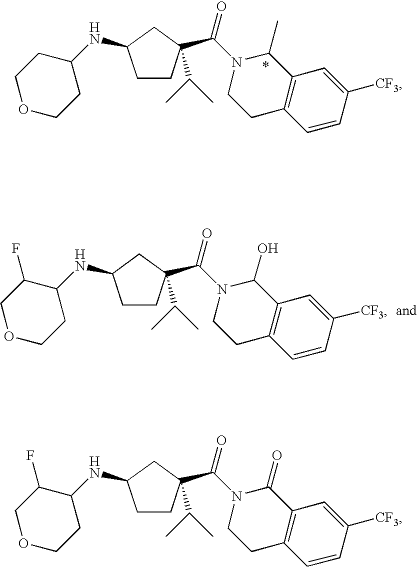
and pharmaceutically acceptable salts thereof and individual diastereomers and enantiomers thereof.

13. A pharmaceutical composition which comprises an inert carrier and the compound of claim 1.

14. The use of the compound of claim 1 for the preparation of a medicament useful in the treatment of an inflammatory and immunoregulatory disorder or disease.

15. The use according to claim 14 wherein said disorder or disease is rheumatoid arthritis.
